(19) United States
(12) Patent Application Publication
(10) Pub. No.: US 2010/0125087 A1
(43) Pub. Date: May 20, 2010

(54) NEW COMPOUNDS 575

(75) Inventors: Jorg HOLENZ, Sodertalje (SE); Sofia KARLSTROM, Sodertalje (SE); Jacob KIHLSTROM, Sodertalje (SE); Karin KOLMODIN, Sodertalje (SE); Laszlo RAKOS, Sodertalje (SE); Peter SODERMAN, Sodertalje (SE); Britt-Marie SWAHN, Sodertalje (SE); Stefan VON BERG, Sodertalje (SE); Fredrik VON KIESERITZKY, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/618,114

(22) Filed: Nov. 13, 2009

Related U.S. Application Data
(60) Provisional application No. 61/114,615, filed on Nov. 14, 2008.

(51) Int. Cl. C07D 471/04; A61K 31/437; A61P 25/28; A61P 25/00; C12N 9/99

(52) U.S. Cl. 514/300; 546/113; 435/184

(57) ABSTRACT

The present invention relates to novel compounds of formula (I) and their pharmaceutical compositions. In addition, the present invention relates to therapeutic methods for the treatment and/or prevention of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

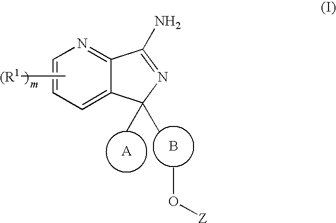

### TECHNICAL FIELD OF THE INVENTION

The present invention relates to novel compounds and their pharmaceutical compositions. In addition, the present invention relates to therapeutic methods for the treatment and/or prevention of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class 1 transmembrane protein called APP, or amyloid precursor protein. Cleavage of APP by BACE generates the extracellular soluble APPβ fragment and the membrane bound CTFβ (C99) fragment that is subsequently cleaved by γ-secretase to generate Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much higher risk of developing AD, and also of developing the disease at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to develop Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of Aβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors, see e.g WO2001/00665, WO2005/058311, WO2006/138265, WO2009005471, WO2009005470, WO2007149033 and WO2009022961.

### OUTLINE OF THE INVENTION

The present invention relates to a compound according to formula (I):

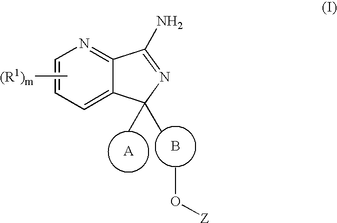

wherein
R¹ is selected from halogen, cyano, NO₂, SO₂R², C₁₋₆alkyl, C₂₋₆alkenyl, C₂₋₆alkynyl, NR³R⁴, OR², C(O)R², C(O)NR³R⁴ and COOR², wherein said C₁₋₆alkyl, C₂₋₆alkenyl or C₂₋₆alkynyl is optionally substituted with one or more R⁷;
R² is C₁₋₆alkyl, C₂₋₆alkenyl or C₂₋₆alkynyl, wherein said C₁₋₆alkyl, C₂₋₆alkenyl or C₂₋₆alkynyl is optionally substituted with one or more R⁷;
R³ and R⁴ are independently selected from hydrogen, C₁₋₆alkyl, C₂₋₆alkenyl, C₂₋₆alkynyl, aryl, heteroaryl, heterocyclyl and carbocyclyl, wherein said C₁₋₆alkyl, C₂₋₆alkenyl, C₂₋₆alkynyl, aryl, heteroaryl, heterocyclyl or carbocyclyl is optionally substituted with one or more R⁷;
or R³ and R⁴ together with the atom they are attached to form a 4 to 7 membered ring;
A is selected from aryl and heteroaryl, wherein said aryl or heteroaryl is optionally substituted with one or more R⁵;
B is aryl or heteroaryl, wherein said aryl or heteroaryl is optionally substituted with one or more R⁶;
Z is selected from aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl and C₂₋₆alkynylheterocyclyl, wherein said aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl or C₂₋₆alkynylheterocyclyl is optionally substituted with one to three R⁷;
R⁵ is selected from halo, cyano, C₁₋₆alkyl, C₁₋₆haloalkyl, C₃₋₆cycloalkyl, OC₁₋₆alkyl and OC₁₋₆alkylaryl, wherein said C₁₋₆alkyl, C₃₋₆cycloalkyl, OC₁₋₆alkyl or OC₁₋₆alkylaryl, is optionally substituted with one to three R⁷;
R⁶ is halogen or cyano;
R⁷ is selected from halogen, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, OH, cyano, C(O)OC₁₋₃alkyl and NR⁸R⁹, wherein said C₁₋₆alkyl, SO₂C₁₋₃alkyl, OC₁₋₃alkyl, OC₁₋₃haloalkyl, C₁₋₃alkylOH, C₁₋₃alkylNR⁸R⁹ or C(O)OC₁₋₃alkyl is optionally substituted with one or more R¹⁰;
R⁸ and R⁹ are independently selected from hydrogen, C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl and carbocyclyl, wherein said C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl or carbocyclyl is optionally substituted with one or more R¹⁰;
or R⁸ and R⁹ together with the atom they are attached to form a 4 to 6 membered ring;
R¹⁰ is selected from halo, C₁₋₃alkyl, OC₁₋₃alkyl and OC₁₋₃haloalkyl;
R¹¹ and R¹² are independently selected from hydrogen, C₁₋₃alkyl and C₁₋₃haloalkyl;
m is 0, 1 or 2;
as a free base or a pharmaceutically acceptable salt thereof.

One embodiment of the present invention relates to a compound of formula (I), wherein

R¹ is selected from halogen, cyano, NO₂, SO₂R², C₁₋₆alkyl, NR³R⁴, OR², C(O)R², C(O)NR³R⁴ and COOR², wherein said C₁₋₆alkyl is optionally substituted with one or more R⁷;
R² is C₁₋₆alkyl, C₂₋₆alkenyl or C₂₋₆alkynyl, wherein said C₁₋₆alkyl, C₂₋₆alkenyl or C₂₋₆alkynyl is optionally substituted with one or more R⁷;
R³ and R⁴ are independently selected from hydrogen, C₁₋₆alkyl, aryl, heteroaryl, heterocyclyl and carbocyclyl, wherein said C₁₋₆alkyl, aryl, heteroaryl, heterocyclyl or carbocyclyl is optionally substituted with one or more R⁷;
or R³ and R⁴ together with the atom they are attached to form a 4 to 7 membered ring;
A is selected from aryl and heteroaryl, wherein said aryl or heteroaryl is optionally substituted with one or more R⁵;
B is aryl or heteroaryl, wherein said aryl or heteroaryl is optionally substituted with one or more R⁶;
Z is selected from aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl and C₂₋₆alkynylheterocyclyl, wherein said aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl or C₂₋₆alkynylheterocyclyl is optionally substituted with one to three R⁷;
R⁵ is selected from halo, cyano, C₁₋₆alkyl, C₁₋₆haloalkyl, C₃₋₆cycloalkyl, OC₁₋₆alkyl and OC₁₋₆alkylaryl, wherein said C₁₋₆alkyl, C₃₋₆cycloalkyl, OC₁₋₆alkyl or OC₁₋₆alkylaryl, is optionally substituted with one to three R⁷;
R⁶ is halogen or cyano;
R⁷ is selected from halogen, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, OH, cyano and C(O)OC₁₋₃alkyl, wherein said C₁₋₆alkyl, SO₂C₁₋₃alkyl, OC₁₋₃alkyl, OC₁₋₃haloalkyl, C₁₋₃alkylOH, C₁₋₃alkylNR⁸R⁹ or C(O)OC₁₋₃alkyl is optionally substituted with one or more R¹⁰;
R⁸ and R⁹ are independently selected from hydrogen, C₁₋₆alkyl, C₁₋₆haloalkyl, C₁₋₃alkylNR¹¹R¹², C₁₋₃alkylOaryl, heteroaryl, heterocyclyl and carbocyclyl, wherein said C₁₋₆alkyl, C₁₋₆haloalkyl, C₁₋₃alkylNR¹¹R¹², C₁₋₃alkylOaryl, heteroaryl, heterocyclyl or carbocyclyl is optionally substituted with one or more R¹⁰;
or R⁸ and R⁹ together with the atom they are attached to form a 4 to 6 membered ring;
R¹⁰ is selected from halo, C₁₋₃alkyl, OC₁₋₃alkyl and OC₁₋₃haloalkyl;
R¹¹ and R¹² are independently selected from hydrogen, C₁₋₃alkyl and C₁₋₃haloalkyl;
m is 0, 1 or 2.

One embodiment of the present invention relates to a compound of formula (I), wherein

R¹ is selected from halogen, cyano, NO₂, SO₂R², C₁₋₆alkyl, NR³R⁴, OR² and C(O)R², wherein said C₁₋₆alkyl is optionally substituted with one or more R⁷;
R² is C₁₋₆alkyl, wherein said C₁₋₆alkyl is optionally substituted with one or more R⁷;
R³ and R⁴ are independently selected from hydrogen, C₁₋₆alkyl, aryl, heteroaryl, heterocyclyl and carbocyclyl, wherein said C₁₋₆alkyl, aryl, heteroaryl, heterocyclyl or carbocyclyl is optionally substituted with one or more R⁷;
A is selected from aryl and heteroaryl, wherein said aryl or heteroaryl is optionally substituted with one or more R⁵;
B is aryl or heteroaryl, wherein said aryl or heteroaryl is optionally substituted with one or more R⁶;
Z is selected from aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl and C₂₋₆alkynylheterocyclyl, wherein said aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl or C₂₋₆alkynylheterocyclyl is optionally substituted with one to three R⁷;
R⁵ is selected from halo, cyano, C₁₋₆alkyl, C₁₋₆haloalkyl, C₃₋₆cycloalkyl, OC₁₋₆alkyl and OC₁₋₆alkylaryl, wherein said C₁₋₆alkyl, C₃₋₆cycloalkyl, OC₁₋₆alkyl or OC₁₋₆alkylaryl, is optionally substituted with one to three R⁷;
R⁶ is halogen;
R⁷ is selected from halogen, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, OH, cyano and C(O)OC₁₋₃alkyl, wherein said C₁₋₆alkyl, SO₂C₁₋₃alkyl, OC₁₋₃alkyl, OC₁₋₃haloalkyl, C₁₋₃alkylOH, C₁₋₃alkylNR⁸R⁹ or C(O)OC₁₋₃alkyl is optionally substituted with one or more R¹⁰;
R⁸ and R⁹ are independently selected from hydrogen, C₁₋₆alkyl, C₁₋₆haloalkyl, C₁₋₃alkylNR¹¹R¹², C₁₋₃alkylOaryl, heteroaryl, heterocyclyl and carbocyclyl, wherein said C₁₋₆alkyl, C₁₋₆haloalkyl, C₁₋₃alkylNR¹¹R¹², C₁₋₃alkylOaryl, heteroaryl, heterocyclyl or carbocyclyl is optionally substituted with one or more R¹⁰;
or R⁸ and R⁹ together with the atom they are attached to form a 4 to 6 membered ring;
R¹⁰ is selected from halo, C₁₋₃alkyl, OC₁₋₃alkyl and OC₁₋₃haloalkyl;
R¹¹ and R¹² are independently selected from hydrogen, C₁₋₃alkyl and C₁₋₃haloalkyl;
m is 0, or 1.

One embodiment of the present invention relates to a compound of formula (I), wherein A is heteroaryl. According to another embodiment of the present invention, wherein said heteroaryl is pyridinyl or pyrimidine.

One embodiment of the present invention relates to a compound of formula (I), wherein A is aryl. According to another embodiment of the present invention, said aryl is phenyl.

One embodiment of the present invention relates to a compound of formula (I), wherein A is not substituted.

One embodiment of the present invention relates to a compound of formula (I), wherein A is substituted with one or more R⁵.

One embodiment of the present invention relates to a compound of formula (I), wherein Z is selected from aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆haloalkyl, C₃₋₆cyclohaloalkyl, C₁₋₆alkylC₃₋₆cyclohaloalkyl and C₁₋₆alkylheterocyclyl.

One embodiment of the present invention relates to a compound of formula (I), wherein Z is selected from aryl, heteroaryl, heterocyclyl, C₃₋₆cycloalkyl, C₁₋₆alkyl, C₁₋₆haloalkyl, C₃₋₆cyclohaloalkyl, C₁₋₆alkylC₃₋₆cyclohaloalkyl, C₁₋₆alkylaryl, C₁₋₆alkylC₃₋₆cycloalkyl, C₁₋₆alkylheteroaryl and C₁₋₆alkylheterocyclyl.

One embodiment of the present invention relates to a compound of formula (I), wherein Z is not substituted.

One embodiment of the present invention relates to a compound of formula (I), wherein Z is substituted with one to three R⁷. According to another embodiment of the present invention, R⁷ is selected from halogen, C₁₋₆alkyl, SO₂C₁₋₃alkyl, OC₁₋₃alkyl, OC₁₋₃haloalkyl and cyano, wherein said C₁₋₆alkyl, SO₂C₁₋₃alkyl, OC₁₋₃alkyl or OC₁₋₃haloalkyl is optionally substituted with one or more R¹⁰.

One embodiment of the present invention relates to a compound of formula (I), wherein R⁶ is fluoro.

One embodiment of the present invention relates to a compound of formula (I), wherein m is 0.

One embodiment of the present invention relates to a compound of formula (I), wherein

A is selected from aryl and heteroaryl, wherein said aryl or heteroaryl is optionally substituted with one or more R⁵;
B is aryl or heteroaryl, wherein said aryl or heteroaryl is optionally substituted with one or more R⁶;
Z is selected from aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl and C₂₋₆alkynylheterocyclyl, wherein said aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl or C₂₋₆alkynylheterocyclyl is optionally substituted with one to three R⁷;
R⁵ is selected from halo, cyano, C₁₋₆alkyl, C₁₋₆haloalkyl, C₃₋₆cycloalkyl, OC₁₋₆alkyl and OC₁₋₆alkylaryl, wherein said C₁₋₆alkyl, C₃₋₆cycloalkyl, OC₁₋₆alkyl or OC₁₋₆alkylaryl, is optionally substituted with one to three R⁷;
R⁶ is halogen;
R⁷ is selected from halogen, C₁₋₆alkyl, OC₁₋₃alkyl, OC₁₋₃haloalkyl and cyano, wherein said C₁₋₆alkyl, OC₁₋₃alkyl or OC₁₋₃haloalkyl is optionally substituted with one or more R¹⁰;
R¹⁰ is halo;
m is 0 or 1.

One embodiment of the present invention relates to a compound of formula (I), wherein

A is heteroaryl, wherein said heteroaryl is optionally substituted with one or more R⁵;
B is aryl;
Z is selected from C₃₋₆cycloalkyl, C₁₋₆alkyl and C₁₋₆alkylC₃₋₆cycloalkyl, wherein said C₃₋₆cycloalkyl, C₁₋₆alkyl or C₁₋₆alkylC₃₋₆cycloalkyl is optionally substituted with one to three R⁷;
R⁵ is selected from C₁₋₆alkyl and OC₁₋₆alkyl, wherein said C₁₋₆alkyl or OC₁₋₆alkyl is optionally substituted with one to three R⁷;
R⁶ is halogen;
R⁷ is halogen;
m is 0.

According to another embodiment of the present invention, B is phenyl.

The present invention also relates to a compound selected from
5-(3-Isobutoxyphenyl)-5-(pyridin-4-yl)-5H-pyrrolo[3,4-b]pyridin-7-amine;
5-(3-(Isopentyloxy)phenyl)-5-(pyridin-4-yl)-5H-pyrrolo[3,4-b]pyridin-7-amine;
5-(3-(Cyclopentylmethoxy)phenyl)-5-(pyridin-4-yl)-5H-pyrrolo[3,4-b]pyridin-7-amine;
5-(3-(Cyclobutylmethoxy)phenyl)-5-(pyridin-4-yl)-5H-pyrrolo[3,4-b]pyridin-7-amine;
5-(3-(2,2-Difluorocyclopropyl)methoxy)phenyl)-5-(pyridin-4-yl)-5H-pyrrolo[3,4-b]pyridin-7-amine;
5-(3-(3-Fluoropropoxy)phenyl)-5-(2-(trifluoromethyl)pyridin-4-yl)-5H-pyrrolo[3,4-b]pyridin-7-amine;
5-(3-(Cyclobutylmethoxy)phenyl)-5-(2-(trifluoromethyl)pyridin-4-yl)-5H-pyrrolo[3,4-b]pyridin-7-amine;
5-(3-(Cyclopentyloxy)phenyl)-5-(pyridin-4-yl)-5H-pyrrolo[3,4-b]pyridin-7-amine;
5-(3-Cyclobutoxyphenyl)-5-(pyridin-4-yl)-5H-pyrrolo[3,4-b]pyridin-7-amine;
5-(3-(3-Fluoropropoxy)phenyl)-5-(pyridin-4-yl)-5H-pyrrolo[3,4-b]pyridin-7-amine;
5-(3-(3-Fluoropropoxy)phenyl)-5-(4-methoxy-3,5-dimethylphenyl)-5H-pyrrolo[3,4-b]pyridin-7-amine;
5-(3-(3-Fluoropropoxy)phenyl)-5-(2-methoxypyridin-4-yl)-5H-pyrrolo[3,4-b]pyridin-7-amine;
5-(3-(3-Fluoropropoxy)phenyl)-5-(2-methylpyridin-4-yl)-5H-pyrrolo[3,4-b]pyridin-7-amine;
5-(3-(3-Fluoropropoxy)phenyl)-5-(4-methoxy-3-(trifluoromethyl)phenyl)-5H-pyrrolo[3,4-b]pyridin-7-amine;
5-(2,6-Dimethylpyridin-4-yl)-5-(3-isobutoxyphenyl)-5Hpyrrolo[3,4-b]pyridin-7-amine acetate;
5-(3-Isobutoxyphenyl)-5-(6-methoxy-5-methylpyridin-3-yl)-5H-pyrrolo[3,4-b]pyridin-7-amine acetate;
5-(3-Isobutoxyphenyl)-5-(6-methoxy-5-methylpyridin-3-yl)-5H-pyrrolo[3,4-b]pyridin-7-amine acetate;
5-(4-(Difluoromethoxy)phenyl)-5-(3-isobutoxyphenyl)-5H-pyrrolo[3,4-b]pyridin-7-amine;
5-(3-Methoxyphenyl)-5-(pyridin-4-yl)-5H-pyrrolo[3,4-b]pyridin-7-amine;
5-(2-(Difluoromethoxy)-6-methylpyridin-4-yl)-5-(3-(3-fluoropropoxy)phenyl)-5H-pyrrolo[3,4-b]pyridin-7-amine;
5-(3-Chloro-4-methoxyphenyl)-5-(3-(3-fluoropropoxy)phenyl)-5H-pyrrolo[3,4-b]pyridin-7-amine;
5-(3-(3,3-Difluoropropoxy)phenyl)-5-(2-(trifluoromethyl)pyridin-4-yl)-5H-pyrrolo[3,4-b]pyridin-7-amine;
5-(3-(3,3-Difluoropropoxy)phenyl)-5-(5-methoxy-4,6-dimethylpyridin-2-yl)-5H-pyrrolo[3,4-b]pyridin-7-amine; and
5-(2-(Difluoromethoxy)pyridin-4-yl)-5-(3-(3-fluoropropoxy)phenyl)-5H-pyrrolo[3,4-b]pyridin-7-amine
   as a free base or a pharmaceutically acceptable salt thereof.

In another aspect of the invention, there is provided a pharmaceutical composition comprising as active ingredient a therapeutically effective amount of a compound according formula (I) in association with pharmaceutically acceptable excipients, carriers or diluents.

In another aspect of the invention, there is provided a compound according to formula (I), or a pharmaceutically acceptable salt thereof, for use as a medicament.

In another aspect of the invention, there is provided use of a compound according to formula (I), as a medicament for treating or preventing an Aβ-related pathology.

In another aspect of the invention, there is provided use of a compound according to formula (I), as a medicament for treating or preventing an Aβ-related pathology, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer Disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided a method of treating or preventing an Aβ-related pathology in a mammal, such as a human, comprising administering to said patient a therapeutically effective amount of a compound according to formula (I), and at least one cognitive enhancing agent, memory enhancing agent, or choline esterase inhibitor, wherein said Aβ-related pathology is Alzheimer Disease.

The present invention relates to the use of compounds of formula (I) as hereinbefore defined as well as to the salts thereof. Salts for use in pharmaceutical compositions will be pharmaceutically acceptable salts, but other salts may be useful in the production of the compounds of formula (I)

It is to be understood that the present invention relates to any and all tautomeric forms of the compounds of formula (I).

Compounds of the invention can be used as medicaments. In some embodiments, the present invention provides compounds of formula (I), or pharmaceutically acceptable salts, tautomers or in vivo-hydrolysable precursors thereof, for use as medicaments. In some embodiments, the present invention provides compounds described here in for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy, traumatic brain injury or cortical basal degeneration.

In some embodiments, the present invention provides use of compounds of formula (I) or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of formula (I), or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of formula (I) or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula (I) or a pharmaceutically acceptable salt, tautomer or in vivo-hydrolysable precursors and a cognitive and/or memory enhancing agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula (I) or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the invention has been diagnosed with a particular disease or disorder, such as those described herein. In these cases, the mammal or human being treated is in need of such treatment. Diagnosis, however, need not be previously performed.

The present invention also includes pharmaceutical compositions, which contain, as the active ingredient, one or more of the compounds of the invention herein together with at least one pharmaceutically acceptable carrier, diluent or excipient.

The definitions set forth in this application are intended to clarify terms used throughout this application. The term “herein” means the entire application.

All compounds in the present invention may exist in particular geometric or stereo isomeric forms. The present invention takes into account all such compounds, including cis- and trans isomers, R- and S-enantiomers, diastereomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in stable compounds.

As used in this application, the term “optionally substituted,” means that substitution is optional and therefore it is possible for the designated atom or moiety to be unsubstituted.

In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example when a substituent is methyl (i.e., CH₃), then 3 hydrogens on the carbon atom can be replaced. Examples of such substituents include, but are not limited to: halo, CN, NH₂, OH, COOH, OC₁₋₆alkyl, C₁₋₆alkylOH, SO₂H, C₁₋₆alkyl, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, N(C₁₋₆alkyl)C(O)C₁₋₆alkyl, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)_{z}, NHC(O)aryl, NarylC(O)aryl, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)_{z}, NHC(O)heteroaryl, NheteroarylC(O)heteroaryl, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NHC(O)C₅₋₆heterocyclyl, NC₅₋₆heterocyclyl C(O)C₅₋₆heterocyclyl.

As used herein, “alkyl”, used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₀₋₆ alkyl” denotes alkyl having 0, 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. In the case where a subscript is the integer 0 (zero) the group to which the subscript refers to indicates that the group may be absent, i.e. there is a direct bond between the groups.

As used herein, “alkenyl” used alone or as a suffix or prefix is intended to include both branched and straight-chain alkene or olefin containing aliphatic hydrocarbon groups having from 2 to 12 carbon atoms or if a specified number of carbon atoms is provided then that specific number would be intended. For example “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl.

As used herein, “alkynyl” used also or as a suffix or prefix is intended to include to include both branched and straight-chain alkynyl or olefin containing aliphatic hydrocarbon groups having from 2 to 12 carbon atoms or if a specified number of carbon atoms is provided then that specific number would be intended. Examples include, but are not limited to, ethynyl, 1-propynyl, 2-propynyl, 3-butynyl, pentynyl, hexynyl and 1-methylpent-2-ynyl.

As used herein, “haloalkyl”, used alone or as a suffix or prefix, is intended to include both branched and straight chain saturated aliphatic hydrocarbon groups, having at least one halogen substituent and having from 1 to 12 carbon atoms or if a specified number of carbon atoms is provided then that specific number would be intended. For example “C₀₋₆haloalkyl” denotes alkyl having 0, 1, 2, 3, 4, 5 or 6 carbon atoms. Examples of haloalkyl include, but are not limited to, fluoromethyl, difluoromethyl, trifluoromethyl, chlorofluoromethyl, 1-fluoroethyl, 3-fluoropropyl, 2-chloropropyl, 3,4-difluorobutyl.

As used herein, “aromatic” refers to hydrocarbonyl groups having one or more unsaturated carbon ring(s) having aromatic characters, (e.g. 4n+2 delocalized electrons) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4n+2 delocalized electrons).

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. Examples of polycyclic rings include, but are not limited to, 2,3-dihydro-1,4-benzodioxine and 2,3-dihydro-1-benzofuran.

As used herein, the term “cycloalkyl” or “carbocyclyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, the term “cycloalkenyl” is intended to include unsaturated ring groups, having the specified number of carbon atoms. These may include fused or bridged polycyclic systems. Preferred cycloalkenyls have from 3 to 10 carbon atoms in their ring structure, and more preferably have 3, 4, 5, and 6 carbons in the ring structure. For example, “C₃₋₆ cycloalkenyl” denotes such groups as cyclopropenyl, cyclobutenyl, cyclopentenyl, or cyclohexenyl.

As used herein, “halo” or “halogen” refers to fluoro, chloro, bromo, and iodo.

“Counterion” is used to represent a small, negatively or positively charged species such as chloride, bromide, hydroxide, acetate, sulfate, tosylate, benezensulfonate, ammonium, lithium ion and sodium ion and the like.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂— group is optionally be replaced by a —C(O)—; and where unless stated to the contrary a ring nitrogen or sulphur atom is optionally oxidised to form the N-oxide or S-oxide(s) or a ring nitrogen is optionally quarternized; wherein a ring —NH is optionally substituted with acetyl, formyl, methyl or mesyl; and a ring is optionally substituted with one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is a non-aromatic heterocycle. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydrofuranyl and 2,5-dioxoimidazolidinyl.

As used herein, “heteroaryl” refers to a heteroaromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, benzoxazolyl, aza-benzoxazolyl indolinyl, imidazothiazolyl and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the non-toxic salts or the quaternary ammonium salts of the parent compound formed, for example, from non-toxic inorganic or organic acids. For example, such non-toxic salts include those derived from inorganic acids such as hydrochloric acid.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like diethyl ether, ethyl acetate, ethanol, isopropanol, or acetonitrile are used.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

Compounds of the invention further include hydrates and solvates.

The present invention further includes isotopically-labelled compounds of the invention. An “isotopically” or “radio-labelled” compound is a compound of the invention where one or more atoms are replaced or substituted with an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labelled compounds will depend on the specific application of that radio-labelled compound. For example, for in vitro receptor labeling and competition assays, compounds that incorporate ³H, ¹⁴C, ⁸²Br, ¹²⁵I, ¹³¹I, ³⁵S, or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labelled compound” is a compound that has incorporated at least one radionuclide. In some embodiments the radionuclide is selected from the group consisting of ³H, ¹⁴C, ¹²⁵I, ³⁵S and ⁸²Br.

For the avoidance of doubt the present invention relates to any one of compounds falling within the scope of formula (I) as defined above.

It will be appreciated that throughout the specification, the number and nature of substituents on rings in the compounds of the invention will be selected so as to avoid sterically undesirable combinations.

The anti-dementia treatment defined herein may be applied as a sole therapy or may involve, in addition to the compound of the invention, conventional therapy. Such therapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Additional conventional therapy may include one or more of the following categories of agents:

(i) antidepressants such as agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine and equivalents and pharmaceutically active isomer(s) and metabolite(s) thereof.

(ii) atypical antipsychotics including for example quetiapine and pharmaceutically active isomer(s) and metabolite(s) thereof.

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof.

(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof

(v) anticonvulsants including for example carbamazepine, valproate, lamotrogine, gabapentin and equivalents and pharmaceutically active isomer(s) and metabolite(s) thereof.

(vi) Alzheimer's therapies including for example donepezil, memantine, tacrine and equivalents and pharmaceutically active isomer(s) and metabolite(s) thereof.

(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof.

(viii) migraine therapies including for example almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof.

(ix) stroke therapies including for example abciximab, activase, NXY-059, citicoline, crobenetine, desmoteplase, repinotan, traxoprodil and equivalents and pharmaceutically active isomer(s) and metabolite(s) thereof.

(x) urinary incontinence therapies including for example darafenacin, falvoxate, oxybutynin, propiverine, robalzotan, solifenacin, tolterodine and equivalents and pharmaceutically active isomer(s) and metabolite(s) thereof.

(xi) neuropathic pain therapies including for example gabapentin, lidoderm, pregablin and equivalents and pharmaceutically active isomer(s) and metabolite(s) thereof.

(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof.

(xiii) insomnia therapies including for example agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon, zolpidem and equivalents and pharmaceutically active isomer(s) and metabolite(s) thereof.

(xiv) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combination products employ the compounds of this invention within the dosage range described herein and the other pharmaceutically active compound or compounds within approved dosage ranges and/or the dosage described in the publication reference.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

### Methods of Preparation

The present invention also relates to processes for preparing the compound of formula (I) as a free base or a pharmaceutically acceptable salt thereof. Throughout the following description of such processes it is to be understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in Protective Groups in Organic Synthesis by T. W. Greene, P. G. M Wutz, 3^{rd} Edition, Wiley-Interscience, New York, 1999. It is understood that microwaves can alternatively be used for the heating of reaction mixtures.

Another aspect of the present invention provides a process for preparing a compound of formula (I), or a pharmaceutically acceptable salt thereof, wherein R¹³ and R¹⁴ are, unless stated otherwise, defined as A or B—OZ in formula (I) above, Z is defined as in formula (I) above; and R¹ is, unless otherwise specified, as defined in formula (I). Said process comprises of:

(i) Formation of a Corresponding Compound of Formula (IV):

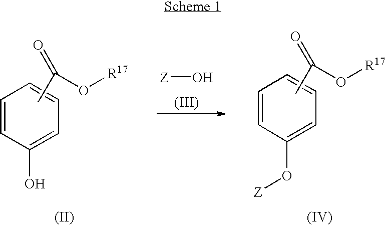

A compound of formula (IV), wherein R¹⁷ is an alkyl (such as methyl or ethyl) may be obtained (Scheme 1), by reacting a compound of formula (II) with a compound of formula (III), wherein Z is defined as for formula (I), using a suitable azodicarboxylate (such as diisopropyl azodicarboxylate or diethyl azodicarboxylate) and triphenylphosphine in a suitable solvent (such as THF or toluene), at a temperature range of 0° C. to r.t.

### (ii) Formation of a Corresponding Compound of Formula (V):

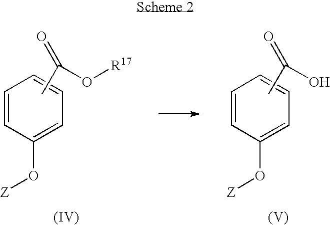

A compound of formula (V) may be prepared by reacting a compound of formula (IV) and an appropriate base (such as sodium hydroxide, potassium hydroxide or lithium hydroxide) in a suitable solvent (such as THF, DMF, water or mixtures thereof), at a temperature range of 0° C. to reflux (Scheme 2).

(iii) Formation of a Corresponding Compound of Formula (VI):

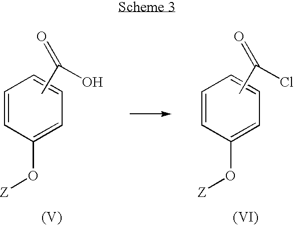

A compound of formula (VI) may be prepared by reacting a compound of formula (V) with an appropriate chlorination reagent such as thionyl chloride or oxalylchloride in a suitable solvent such as dichloromethane or dichloro ethane, at 0° C. to r.t. (Scheme 3).

### (iv) Formation of a Corresponding Compound of Formula (X)

A compound of formula (X) may be obtained as depicted in Scheme 4 for example by metallation or halogen metal exchange of a compound of formula (VII), wherein G is either hydrogen or halogen, to obtain an intermediate of formula (VIII), wherein L is a ligand such as halogen and n is between 0 and 6. The intermediate (VIII) is not isolated but reacted further with a compound of formula (IX), wherein LG is either N(CH₃)(OCH₃) or halogen or another suitable leaving group as described by, for example, R. K. Dieter, (Tetrahedron, 55 (1999) 4177-4236).

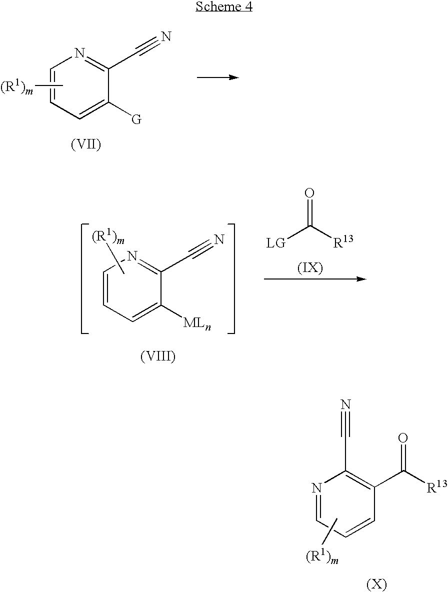

Said reaction may be carried out by reacting a compound of formula (VII) with an appropriate metallating reagent, such as a lithium reagent (such as tert-butyllithium, n-butyllithium, lithium diispropylamide or lithium tetramethyl piperidine) or with a Grignard reagent (such as isopropylmagnesium bromide) or with a metal, such as magnesium, zinc or manganese by standard methods known in the art. Optionally, the formed intermediate of formula (VIII) may be further transmetallated by treatment with a metal salt or metal complex, such as copper cyanide di(lithium bromide), to obtain a new intermediate of formula (VIII), and then treat said intermediate of formula (VIII) with a compound of formula (IX), wherein LG represents a leaving group such as a halogen (such as chlorine) or N(CH₃)(OCH₃). Optionally, this transformation may be performed under the influence of a transition metal catalyst such as a palladium salt or complex. as described in for example R. K. Dieter, (Tetrahedron, 55 (1999) 4177-4236). The reaction is performed in a suitable solvent, such as diethyl ether or tetrahydrofuran, at a temperature between −105° C. and room temperature

### (v) Formation of a Corresponding Compound of Formula (XIV):

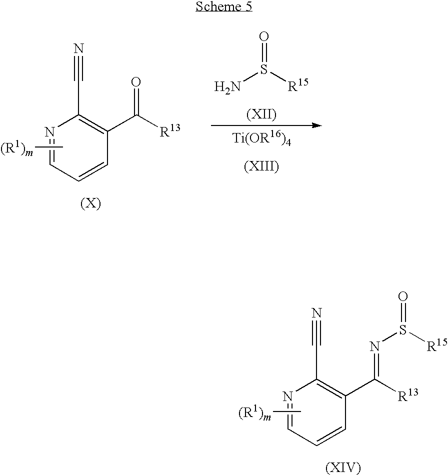

A compound of formula (XIV) may be obtained by reacting a compound of formula (X) with a compound of formula (XII) (Scheme 5), wherein R¹⁵ is alkyl (such as for example tert-butyl) under the influence of a suitable Lewis acid of formula (XIII), wherein R¹⁶ is alkyl (such as ethyl or isopropyl). The reaction is performed in a suitable solvent (such as diethyl ether or tetrahydrofuran) at a temperature between room temperature and reflux temperature.

### (vi) Formation of a Corresponding Compound of Formula (XVI)

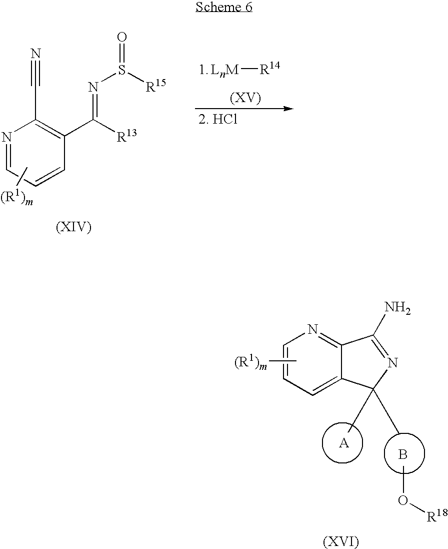

A compound of formula (XVI), wherein R¹⁸ is defined as an alkyl such as methyl, may be prepared as shown in Scheme 6 by treating a compound of formula (XIV), with an appropriate organo metallic reagent of formula (XV), wherein M is a metal (such as lithium zinc or magnesium), L is a ligand (such as halogen) and n is between 0 and 2, and R¹⁴ is as defined above, followed by treatment with a suitable acid, such as hydrochloric acid. The reaction may be performed in a suitable solvent, such as diethyl ether or tetrahydrofuran, at a temperature between −105° C. and room temperature. The organo metallic reagent of formula (XV) may be generated from the corresponding LG-R¹⁴, wherein LG represents a leaving group such as a halogen, such as iodide, bromide or chloride, by known methods as described in Advanced Organic Chemistry by Jerry March 4^{th} edition, Wiley Interscience,

(vii) Formation of a Corresponding Compound of Formula (XVIII)

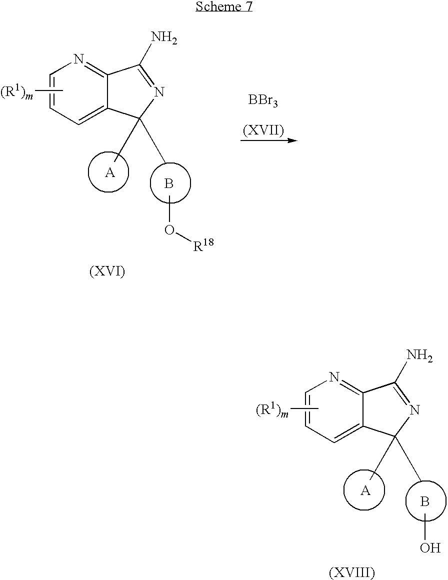

A compound of formula (XVIII) can be obtained, as shown in Scheme 7, by reacting a compound of formula (XVI), wherein R¹⁸ is defined as an alkyl, such as methyl or ethyl, with a reagent of formula (XVII), such as boron tribromide, in a suitable solvent (such as dichloromethane), at a temperature between 0° C. and room temperature.

(viii) Formation of a Corresponding Compound of Formula (XIX)

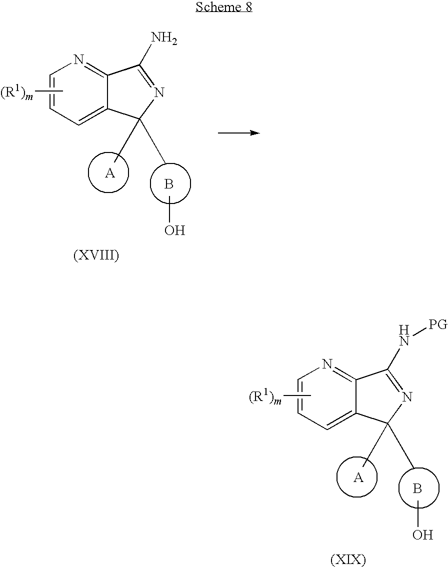

A compound of formula (XIX), wherein PG is a suitable protecting group such as t-butoxycarbonyl, can be obtained, as shown in Scheme 8, by reacting a compound of formula (XVIII) with a suitable reagent (such as di-tert-butyl dicarbonate) mediated by a suitable base, such as 4-dimethylaminopyridine, in a suitable solvent such as THF. A compound of formula (XIX) may also be obtained with other protecting groups (PG) described in Protective Groups in Organic Synthesis by T. W. Greene, P. G. M Wutz, 3^{rd} Edition, Wiley-Interscience, New York, 1999.

### (ix) Formation of a Corresponding Compound of Formula (I)

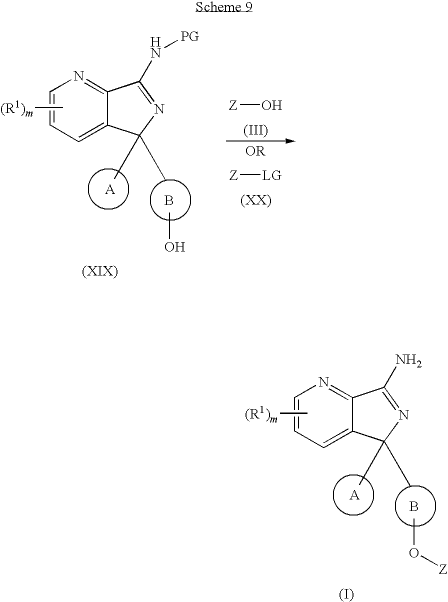

A compound of formula (I) may be obtained (Scheme 9), by reacting a compound of formula (XIX) with a compound of formula (III), wherein Z is defined as for formula (I) above, together with a suitable azodicarboxylate (such as diisopropyl azodicarboxylate or diethyl azodicarboxylate) and triphenylphosphine in a suitable solvent (such as THF or toluene), at a temperature range of 0° C. to r.t. A compound of formula (I) may also be obtained by reacting a compound of formula (XIX) with a compound of formula (XX), wherein Z is defined as for formula (I) above and LG represents a leaving group, such as halogen (such as bromide or iodide) in the presence of a suitable base (such as potassium carbonate or cesium carbonate), in a suitable solvent (such as, N,N-dimethylacetamide or N,N-dimethylformamide).

### (x) Formation of a Corresponding Compound of Formula (I)

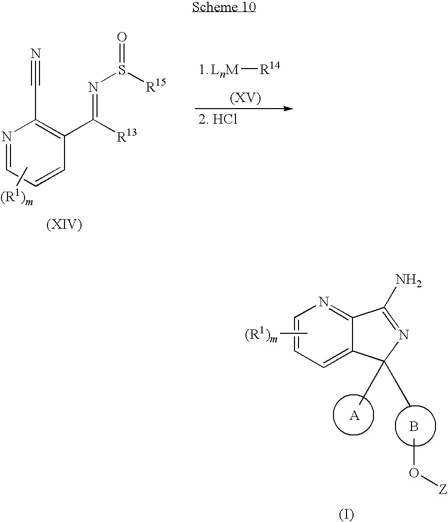

A compound of formula (I) may be prepared by treating a compound of formula (XIV), with an appropriate organo metallic reagent of formula (XV), wherein M is a metal (such as lithium zinc or magnesium), L is a ligand (such as halogen) and n is between 0 and 2, and R¹⁴ is as defined above, followed by treatment with a suitable acid, such as hydrochloric acid. The reaction may be performed in a suitable solvent, (such as diethyl ether or tetrahydrofuran), at a temperature between −105° C. and room temperature. The organo metallic reagent of formula (XV) may be generated from the corresponding LG-R¹⁴, wherein LG represents a leaving group, such as a halogen (such as iodide, bromide or chloride) by known methods as described in for example Advanced Organic Chemistry by Jerry March 4^{th} edition, Wiley Interscience.

Compounds of formula (II), (III), (VII), (IX), (XII), (XIII), (XV), (XVII), and (XX) are commercially available compounds, or they are known in the literature, or they are prepared by standard processes known in the art.

### General Methods

All solvents used were of analytical grade and commercially available anhydrous solvents were routinely used for reactions.

Starting materials used were available from commercial sources, or prepared according to literature procedures.

Microwave heating was performed in a Creator, Initiator or Smith Synthesizer Single-mode microwave cavity producing continuous irradiation at 2450 MHz.

¹H NMR spectra were recorded in the indicated deuterated solvent at 400 MHz. The 400 MHz spectra were obtained unless stated otherwise, using a Bruker av400 NMR spectrometer equipped with a 3 mm flow injection SEI ¹H/D-¹³C probe head with Z-gradients, using a BEST 215 liquid handler for sample injection, or using a Bruker DPX400 NMR spectrometer equipped with a 4-nucleus probehead with Z-gradients. 500 MHz spectra were recorded using a Bruker 500 MHz Avance III NMR spectrometer, operating at 500 MHz for ¹H, 125 MHz for ¹³C, and 50 MHz for ¹⁵N equipped with a 5 mm TXI probehead with Z-gradients. 600 MHz spectra were recorded using a Bruker DRX600 NMR spectrometer, operating at 600 MHz for ¹H, 150 MHz for ¹³C, and 60 MHz for ¹⁵N equipped with a 5 mm TXI probehead with Z-gradients

Chemical shifts are given in ppm down- and upheld from TMS. Resonance multiplicities are denoted s, d, t, q, m and br for singlet, doublet, triplet, quartet, multiplet, and broad respectively. In cases where the NMR spectra are complex; only diagnostic signals are reported.

LC-MS analyses were recorded on a Waters LCMS equipped with a Waters X-Terra MS, C8-column, (3.5 um, 100 mm×3.0 mm i.d.). The mobile phase system consisted of A: 10 mM ammonium acetate in water/acetonitrile (95:5) and B: acetonitrile. A linear gradient was applied running from 0% to 100% B in 4-5 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700. Alternative, LC-MS HPLC conditions were as follows: Column: Agilent Zorbax SB-C8 2 mm ID×50 mm Flow: 1.4 mL/minGradient: 95% A to 90% B over 3 min. hold 1 minute ramp down to 95% A over 1 minute and hold 1 minute. Where A=2% acetonitrile in water with 0.1% formic acid and B=2% water in acetonitrile with 0.1% formic acid. UV-DAD 210-400 nm. Or LC-MS analyses were performed on a LC-MS consisting of a Waters sample manager 2777C, a Waters 1525μ binary pump, a Waters 1500 column oven, a Waters ZQ single quadrupole mass spectrometer, a Waters PDA2996 diode array detector and a Sedex 85 ELS detector. The mass spectrometer was configured with an atmospheric pressure chemical ionisation (APCI) ion source which was further equipped with atmospheric pressure photo ionisation (APPI) device. The mass spectrometer scanned in the positive mode, switching between APCI and APPI mode. The mass range was set to m/z 120-800 using a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode. Separation was performed using a Gemini column C18, 3.0 mm×50 mm, 3 μm, (Phenomenex) and run at a flow rate of 1 ml/min. A linear gradient was used starting at 100% A (A: 10 mM ammonium acetate in 5% methanol) and ending at 100% B (methanol). The column oven temperature was set to 40° C.

Mass spectra (MS) were run using an automated system with atmospheric pressure chemical (APCI or CI) or electrospray (+ESI) ionization. Generally, only spectra where parent masses are observed are reported. The lowest mass major ion is reported for molecules where isotope splitting results in multiple mass spectral peaks (for example when chlorine is present).

UPLCMS analyses were performed on an Waters Acquity HPLC system consisting of a Acquity Autosampler, Acquity Sample Organizer, Acquity Column Manager, Acquity Binary Solvent Manager, Acquity UPLC PDA detector and a Waters SQ Detector.

The mass spectrometer was equipped with an electrospray ion source (ES) operated in positive and negative ion mode. The capillary voltage was set to 3.0 kV and the cone voltage to 30 V, respectively. The mass spectrometer was scanned between m/z 100-600 with a scan time of 0.105 s. The diode array detector scanned from 200-400 nm. The temperature of the Column Manager was set to 60° C. Separation was performed on a Acquity column, UPLC BEH, C18 1.7 μM run at a flow rate of 0.5 ml/min. A linear gradient was applied starting at 100% A (A: 10 mM NH₄OAc in 5% CH3CN) ending at 100% B (B: CH3CN) after 1.3 min then 100% B for 0.6 min.

Acquity column, UPLC BEH, C18 1.7 μM. Linear gradient, flow 0.5 ml/min.

0-100% B (MeCN) in 1.3 min, then 100% B for 0.6 min. ESpos/ESneg, m/z 100-600. A (A: 10 mM NH₄OAc in 5% CH3CN)

Acquity column, UPLC BEH, C18 1.7 μM. Linear gradient, flow 0.5 ml/min, 0-100% B (MeCN) in 2.5 min, then 100% B until 3.8 min. ES+/ES−, m/z 100-600.

A (A: 10 mM NH₄OAc in 5% CH3CN)

GC-MS analyses were performed on a Agilent 6890N GC equipped with a Chrompack CP-Sil 5CB column (25 m×0.25 mm i.d. df=0.25)), coupled to an Agilent 5973 Mass Selective Detector operating in a chemical ionization (CI) mode and the MS was scanned between m/z 50-500.

Accurate mass analyses were performed on a QTOF micro (Waters). The mass spectrometer was equipped with an electrospray ionsource that uses two probes, a sample probe and a lock mass probe, respectively. The lock mass solution was Leucine Enkephaline (0.5 ng/uL in MilliQ water) infused at flow rate of 0.1 mL/min. The reference scan frequency was set to 5.5 s. Before the analysis, the mass spectrometer was calibrated in the positive mode between 90-1000 Da using a solution of NaFormate. The mass spectrometer scanned in the centroid mode between m/z 100-1000 with a scan time of 1.0 s. The capillary voltage was set to 3.3 kV and the ES cone voltage was set to 28 V. The source temperature and desolvation temperature were set to 110° C. and 350° C., respectively. The collision energy was set to 6.0 V. The QTOF micro was equipped with an LC (HP1100 Agilent, Degasser, Binary pump, ALS and a column compartment). The column used was a Gemini C18, 3.0×50 mm, 3 u run at a flowrate of 1.0 mL/min. A linear gradient was applied starting at 100% A (A: 10 mM ammonium acetate) and ending at 100% B (B: acetonitrile) after 4 min. The column oven temperature was set to 40° C. The flow was split 1:4 prior to the ion source. 3 μL of the sample was injected on the column.

HPLC assays were performed using an Agilent HP 1100 Series system equipped with a Waters X-Terra MS, C₈ column (3.0×100 mm, 3.5 μm). The column temperature was set to 40° C. and the flow rate to 1.0 mL/min. The Diode Array Detector was scanned from 200-300 nm. A linear gradient was applied, run from 0% to 100% B in 4 min. Mobile phase A: 10 mM ammonium acetate in water/acetonitrile (95:5), mobile phase B: acetonitrile.

Preparative HPLC was performed on a Waters Auto purification HPLC-UV system with a diode array detector using a Waters XTerra MS C₈ column (19×300 mm, 7 μm) and a linear gradient of mobile phase B was applied. Mobile phase A: 0.1 M ammonium acetate in water/acetonitrile (95:5) and mobile phase B: acetonitrile. Flow rate: 20 mL/min.

Thin layer chromatography (TLC) was performed on Merck TLC-plates (Silica gel 60 F₂₅₄) and spots were UV visualized. Flash chromatography was performed using Merck Silica gel 60 (0.040-0.063 mm), or employing a Combi Flash® Companion™ system using RediSep™ normal-phase flash columns.

Room temperature refers to 20-25° C.

Solvent mixture compositions are given as volume percentages or volume ratios.

### Terms and Abbreviations:

Atm atmospheric pressure;
Boc t-butoxycarbonyl;
Cbz benzyl-oxy-carbonyl;
DCM dichloromethane;
DIPEA diisopropylethylamine;
DMF N;N-dimethyl formamide;
DMSO dimethyl sulfoxide;
Et₂O diethyl ether;
EtOAc ethyl acetate;
h hour(s);
HPLC high pressure liquid chromatography;

### MeOH Methanol;

min minute(s);
NMR nuclear magnetic resonance;
Psi pounds per square inch;
TFA trifluoroacetic acid;
THF tetrahydrofuran;
ACN acetonitrile.
r.t. room temperature
sat saturated
aq aqueous

Compounds have been named using CambridgeSoft MedChem ELN v2.1 or ACD/Name, version 9.0, software from Advanced Chemistry Development, Inc. (ACD/Labs), Toronto ON, Canada, www.acdlabs.com, 2004.

### EXAMPLES

Below follows a number of non-limiting examples of compounds of the invention.

### Example 1i

### 3-(3-Fluoropropoxy)benzoic acid

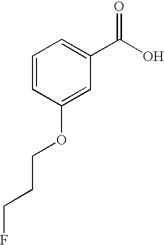

Methyl 3-hydroxybenzoate (10 g, 65.73 mmol), triphenylphosphine (18.96 g, 72.30 mmol) and 3-fluoropropan-1-ol (5.43 mL, 72.30 mmol) were dissolved in THF (100 mL) and cooled to 0° C. Diisopropyl azodicarboxylate (14.23 mL, 72.30 mmol) was added and the mixture was stirred at rt for 1 h. Water was added and the mixture was concentrated. Diethyl ether was added to the residue and the organic phase was washed with NaOH (2M) and brine, dried over MgSO₄ and concentrated. The residue was dissolved in THF (50 mL). Sodium hydroxide (3M aq.) (54.8 mL, 164.31 mmol) was added and the mixture was heated to 50° C. for 2 h. The reaction mixture was washed with DCM and the water phase was acidified with. HCl (conc) until pH ˜2 and then extracted with DCM. The organic phase was dried over MgSO₄, filtered and concentrated to give 12.8 g (98% yield) of the title compound:

¹H NMR (500 MHz, DMSO-d₆) δ ppm 2.06-2.17 (m, 2H) 4.12 (t, 2H) 4.57 (t, 1H) 4.66 (t, 1H) 7.18-7.23 (m, 1H) 7.39-7.46 (m, 2H) 7.51-7.56 (m, 1H) 13.02 (s, 1H). MS (ES) m/z 197 [M−1]⁻.

### Example 2i

### 3-(3-Fluoropropoxy)benzoyl chloride

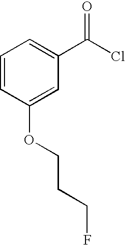

To a suspension of 3-(3-fluoropropoxy)benzoic acid (5.26 g, 26.54 mmol) in anhydrous dichloromethane (50 mL) was added oxalyl chloride (2.3 mL, 26.5 mmol), followed by addition of anhydrous DMF (0.5 mL). The reaction mixture was stirred at room temperature for 3 h and concentrated in vacuo to give the crude title compound in quantitative yield, which was used directly in the next step without further purification. An analytical sample was treated with methanol to generate 3-(3-fluoro-propoxy)-benzoic acid methyl ester:

MS (ES−) m/z 211 [M−H]⁻.

### Example 3i

### 3-(3-Methoxybenzoyl)picolinonitrile

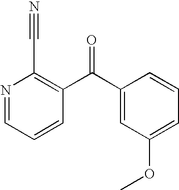

3-Bromopicolinonitrile (2.8 g, 15.30 mmol) in dry THF (50 mL) was added dropwise over 1.5 h to a bottle of Rieke® Zinc (50.0 mL, 38.25 mmol) under nitrogen atmosphere and stirred for 1 h at r.t. The reaction mixture was cooled to −20° C. and stirred for 22 h. The excess zinc was removed by decantation, and the solution was cooled to −20° C. CuCN (LiBr)₂ (1M in THF) (15.30 mL, 15.30 mmol) was added to the solution. The reaction mixture was allowed to reach 0° C. and stirred for 30 min. The mixture was cooled to −40° C. and 3-methoxybenzoyl chloride (2.26 mL, 16.1 mmol) was added. The reaction mixture was allowed to reach r.t. over night. Aqueous NH₄Cl (sat.) was added and the mixture was extracted with EtOAc. The organic phase was washed with NaHCO₃ (sat.) and brine, dried over MgSO₄ and concentrated. Column chromatography using a gradient of 0-40% EtOAc in n-heptane gave 2.2 g (60% yield) of the title compound:

¹H NMR (500 MHz, DMSO-d₆) δ ppm 8.94-8.97 (m, 1H), 8.20-8.24 (m, 1H), 7.87-7.91 (m, 1H), 7.50-7.54 (m, 1H), 7.32-7.38 (m, 3H), 3.83 (s, 3H);

### Example 4i

### 3-(3-(3-Fluoropropoxy)benzoyl)picolinonitrile

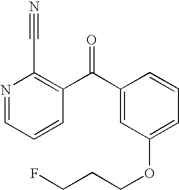

The title compound was synthesized as described for Example 31 in 33% yield starting from 3-bromopicolinonitrile (3.5 g, 19.13 mmol) and 3-(3-fluoropropoxy)benzoyl chloride (4.35 g, 20.08 mmol):

¹H NMR (500 MHz, DMSO-d₆) δ ppm 8.93-8.97 (m, 1H), 8.20-8.24 (m, 1H), 7.87-7.91 (m, 1H), 7.49-7.54 (m, 1H), 7.34-7.39 (m, 3H), 4.66 (t, 1H), 4.57 (t, 1H), 4.15 (t, 2H), 2.07-2.17 (m, 2H).

### Example 5i

### 3-(3-Isobutoxybenzoyl)picolinonitrile

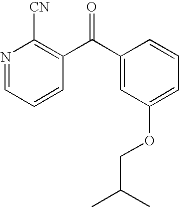

The title compound was synthesized as described for Example 31 in 26% yield starting from 3-isobutoxybenzoyl chloride (3.5 g, 16.4 mmol) and 3-bromopicolinonitrile (3 .g, 16.4 mmol):

¹H NMR (500 MHz, DMSO-d₆) δ ppm 8.94-8.98 (m, 1H) 8.20-8.24 (m, 1H) 7.87-7.92 (m, 1H) 7.48-7.53 (m, 1H) 7.30-7.37 (m, 3H) 3.82 (d, 2H) 1.98-2.07 (m, 1H) 0.98 (d, 6H).

### Example 6i

### N-((2-Cyanopyridin-3-yl)(3-methoxyphenyl)methylene)-2-methylpropane-2-sulfinamide

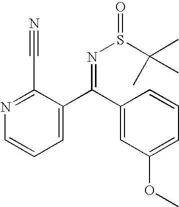

2-Methyl-2-propanesulfinamide (1.824 g, 15.05 mmol) was added to a mixture of titanium(IV) ethoxide (7.17 mL, 34.21 mmol) and 3-(3-methoxybenzoyl)picolinonitrile (3.26 g, 13.68 mmol) in THF (60 mL). The reaction mixture was heated to reflux and stirred for 42 h. MeOH (7 mL), NaHCO₃ (sat, 0.2 ml) and EtOAc was added and the slurry was filtered through celite and MgSO₄ and then concentrated. Column chromatography using a gradient of 0-45% EtOAc in heptane gave 3.22 g (69% yield) of the title compound.

¹H NMR (500 MHz, DMSO-d₆) δ ppm 8.78-8.84 (m, 1H), 7.97-8.22 (m, 1H), 7.76-7.88 (m, 1H), 7.42 (t, 1H), 7.19-7.25 (m, 1H), 7.10-7.14 (m, 1H), 6.94-7.00 (m, 1H), 3.77 (s, 3H), 1.23-1.30 (m, 9H). MS (ES+) m/z 342 [M+1]⁺.

### Example 7i

### N-((2-Cyanopyridin-3-yl)(3-(3-fluoropropoxy)phenyl)methylene)-2-methylpropane-2-sulfinamide

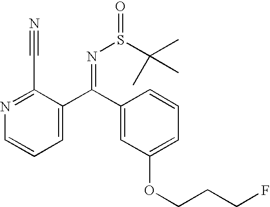

The title compound was synthesized as described for Example 61 in 33% yield starting from 3-(3-(3-fluoropropoxy)benzoyl)picolinonitrile (1.81 g, 6.37 mmol) and 2-methyl-2-propanesulfinamide (0.849 g, 7.00 mmol):

¹H NMR (500 MHz, DMSO-d₆) δ ppm 8.80-8.86 (m, 1H), 8.00-8.21 (m, 1H), 7.77-7.89 (m, 1H), 7.44 (t, 1H), 7.23-7.28 (m, 1H), 7.09-7.13 (m, 1H), 7.00-7.06 (m, 1H), 4.64 (t, 1H), 4.55 (t, 1H), 4.07-4.12 (m, 2H), 2.04-2.16 (m, 2H), 1.24-1.32 (m, 9H); MS (ES+) m/z 388 [M+1]⁺.

### Example 8i

### (N-((2-Cyanopyridin-3-yl)(3-isobutoxyphenyl)methylene)-2-methylpropane-2-sulfinamide

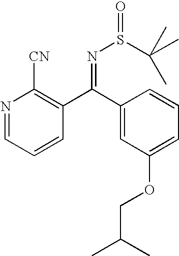

The title compound was synthesized as described for Example 61 in 95% yield starting from 3-(3-isobutoxybenzoyl)picolinonitrile (1.19 g, 4 3 mmol) and 2-methyl-2-propanesulfinamide (0.67 g, 5.5 mmol):

¹H NMR (500 MHz, DMSO-d₆) δ ppm 8.83 (d, 1H) 7.98-8.28 (m, 1H) 7.77-7.90 (m, 1H) 7.42 (t, 1H) 7.21-7.27 (m, 1H) 7.08-7.13 (m, 1H) 7.00 (d, 1H) 3.74-3.80 (m, 2H) 1.96-2.05 (m, 1H) 1.25-1.32 (m, 9H) 0.96 (d, 6H). MS (ES) m/z 384 [M+1]⁺.

### Example 91

### 5-(3-Methoxyphenyl)-5-(pyridin-4-yl)-5H-pyrrolo[3,4-b]pyridin-7-amine

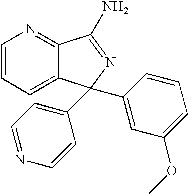

N-((2-Cyanopyridin-3-yl)(3-methoxyphenyl)methylene)-2-methylpropane-2-sulfinamide (1.1 g, 3.22 mmol) in tetrahydrofuran (10 mL) was added to a mixture of 4-iodopyridine (0.859 g, 4.19 mmol) and tert-butyllithium (5.24 mL, 8.38 mmol) in tetrahydrofuran (43 mL). The resulting reaction mixture was stirred at −100° C. for 40 min. The reaction mixture was then allowed to reach r.t slowly. Water was added and the mixture was extracted with DCM. The organic phase was washed with brine, concentrated and dissolved in methanol (20 mL). Hydrogen chloride (1M in diethyl ether) (6.44 mL, 6.44 mmol) was added and the mixture was stirred at r.t. for 3 h and then concentrated. EtOAc and NaHCO₃ (sat) was added to the remaining residue. The organic phase was collected, dried over MgSO₄ and concentrated. Purification by column chromatography using a gradient of 0-5% MeOH (7N NH₃) in DCM gave the title compound (0.88 g, 86% yield).

¹H NMR (500 MHz, DMSO-d₆) δ ppm 8.63-8.67 (m, 1H), 8.43-8.47 (m, 2H), 8.29-8.33 (m, 1H), 7.46-7.50 (m, 1H), 7.28-7.32 (m, 2H), 7.21 (t, 1H), 6.87-6.96 (m, 3H), 6.84-6.87 (m, 1H), 6.80-6.83 (m, 1H), 3.67 (s, 3H); MS (ES+) m/z 317 [M+1]⁺.

### Example 10i

### 5-(3-Methoxyphenyl)-5-(2-(trifluoromethyl)pyridin-4-yl)-5H-pyrrolo[3,4-b]pyridin-7-amine

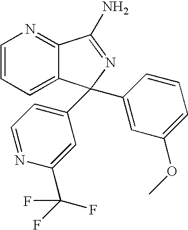

The title compound was synthesized as described for Example 9i in 26% yield starting from N-((2-cyanopyridin-3-yl)(3-methoxyphenyl)methylene)-2-methylpropane-2-sulfinamide (750 mg, 2.20 mmol) and 4-bromo-2-(trifluoromethyl)pyridine (695 mg, 3.08 mmol):

¹H NMR (500 MHz, DMSO-d₆) δ ppm 8.66-8.70 (m, 2H), 8.41-8.45 (m, 1H), 7.72-7.74 (m, 1H), 7.66-7.69 (m, 1H), 7.50-7.53 (m, 1H), 7.21-7.26 (m, 1H), 7.05 (br. s., 2H), 6.90-6.94 (m, 1H), 6.83-6.87 (m, 2H), 3.68 (s, 3H);

MS (ES) m/z 433, 485 [M+1]⁺

### Example 11i

### 3-(7-Amino-5-(pyridin-4-yl)-5H-pyrrolo[3,4-b]pyridin-5-yl)phenol

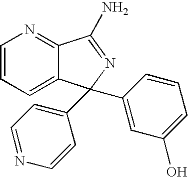

5-(3-Methoxyphenyl)-5-(pyridin-4-yl)-5H-pyrrolo[3,4-b]pyridin-7-amine (0.88 g, 2.78 mmol) was dissolved in DCM (70 mL) and cooled to 0° C. Boron tribromide (0.789 mL, 8.35 mmol) was added and the mixture was stirred at 0° C. for 2 h, the reaction mixture was allowed to reach rt and stirring was continued for 4 h. NH₄OH(konc) and MeOH was added and the pH was adjusted to ˜7-8 using HCl (2M) and NH₄OH (conc.). The mixture was extracted with EtOAc and the organic phase was dried over MgSO₄, filtered and concentrated, to afford the title compound in quantitative yield. The title compound was used in the next step without further purification. MS (ES+) m/z 303 [M+1]⁺.

### Example 12i

### 3-(7-Amino-5-(2-(trifluoromethyl)pyridin-4-yl)-5H-pyrrolo[3,4-b]pyridin-5-yl)phenol

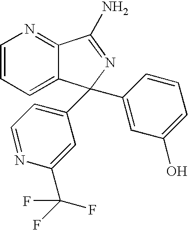

The title compound was synthesized as described for Example 11i in quantitative yield starting from 5-(3-methoxyphenyl)-5-(pyridin-4-yl)-5H-pyrrolo[3,4-b]pyridin-7-amine (0.76 g, 2.40 mmol): MS (ES+) m/z 371 [M+1]⁺.

### Example 13i

### 3-(7-Amino-5-(2,6-dimethylpyridin-4-yl)-5H-pyrrolo[3,4-b]pyridin-5-yl)phenol

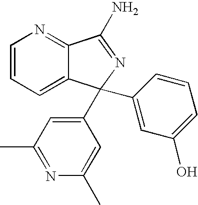

The title compound was synthesized as described for Example 11i in 99% yield starting from 5-(2,6-dimethylpyridin-4-yl)-5-(3-methoxyphenyl)-5H-pyrrolo[3,4-b]pyridin-7-amine (650 mg, 1.89 mmol): MS (ES+) m/z 331 [M+1]⁺.

### Example 14i

### tert-Butyl 5-(3-hydroxyphenyl)-5-(pyridin-4-yl)-5H-pyrrolo[3,4-b]pyridin-7-ylcarbamate

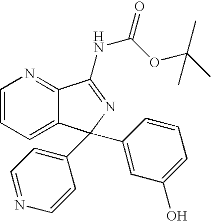

Di-tert-butyl dicarbonate (1.470 g, 6.74 mmol) was added to a mixture of 3-(7-amino-5-(pyridin-4-yl)-5H-pyrrolo[3,4-b]pyridin-5-yl)phenol (0.97 g, 3.21 mmol) and 4-dimethylaminopyridine (0.039 g, 0.32 mmol) in THF (25 mL) and the mixture was stirred over night. Brine and water was added and the mixture was extracted with EtOAc. The organic phase was dried over MgSO₄ and concentrated. The residue was dissolved in methanol (30 mL) and ammonia (conc.) (20 mL) and heated to 50° C. over night. The mixture was cooled to rt, and concentrated. NH₄Cl (sat.) was added and the mixture was extracted with EtOAc. The organic phase was dried over MgSO₄ and concentrated. Purification by column chromatography using 10-100% EtOAc in heptane gave the title compound (0.52 g, 40% yield).

¹H NMR (500 MHz, DMSO-d₆) δ ppm 1.43-1.51 (m, 9H) 6.56-6.76 (m, 3H) 7.03-7.18 (m, 1H) 7.31-7.41 (m, 2H) 7.50-7.66 (m, 1H) 8.13-8.40 (m, 1H) 8.44-8.59 (m, 2H) 8.65-8.82 (m, 1H) 9.37-9.60 (m, 1H) 9.67-10.59 (m, 1H). MS (ES) m/z 403 [M+1]⁺.

### Example 15i

### tert-Butyl 5-(3-hydroxyphenyl)-5-(2-(trifluoromethyl)pyridin-4-yl)-5H-pyrrolo[3,4-b]pyridin-7-ylcarbamate

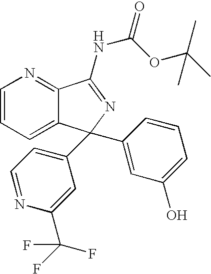

The title compound was synthesized as described for Example 14i in 50% yield starting from 3-(7-amino-5-(2-(trifluoromethyl)pyridin-4-yl)-5H-pyrrolo[3,4-b]pyridin-5-yl)phenol (0.35 g, 0.95 mmol) and di-tert-butyl dicarbonate (0.454 g, 2.08 mmol), except that the reaction mixture was stirred at r.t for 3 weeks.

¹H NMR (500 MHz, DMSO-d₆) δ ppm 9.87 (s, 1H), 9.50 (s, 1H), 8.68-8.77 (m, 2H), 8.44-8.55 (m, 1H), 7.72-7.87 (m, 2H), 7.56-7.61 (m, 1H), 7.07-7.17 (m, 1H), 6.58-6.73 (m, 3H), 1.50 (s, 9H); MS (ES+) m/z 471 [M+1

### Example 16i

### tert-Butyl 5-(2,6-dimethylpyridin-4-yl)-5-(3-hydroxyphenyl)-5H-pyrrolo[3,4-b]pyridin-7-ylcarbamate

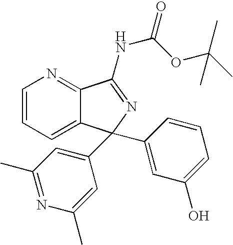

The title compound was synthesized as described for Example 14i in 56% yield starting from 3-(7-amino-5-(2,6-dimethylpyridin-4-yl)-5H-pyrrolo[3,4-b]pyridin-5-yl)phenol (0.62 g, 1.88 mmol) and di-tert-butyl dicarbonate (0.901 g, 4.13 mmol).

¹H NMR (500 MHz, DMSO-d₆) δ ppm 9.66-9.76 (m, 1H), 9.38-9.46 (m, 1H), 8.65-8.73 (m, 1H), 8.28-8.39 (m, 1H), 7.49-7.59 (m, 1H), 7.05-7.18 (m, 1H), 6.98-7.04 (m, 2H), 6.57-6.75 (m, 3H), 2.34-2.40 (m, 6H), 1.46-1.52 (m, 9H); MS (ES+) m/z 431 [M+1]⁺.

### Example 17i

### 4-Bromo-2-difluoromethoxy-6-methyl-pyridine

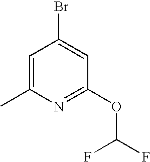

A mixture of 2,4-dihydroxy-6-methyl-pyridine (10.0 g, 79.9 mmol), phosphorous oxybromide (15.35 g, 53.55 mmol) and dimethylformamide (10 mL) was heated to 110° C. for 1 h. The mixture was cooled to r.t. and water (15 mL) was added dropwise, followed by 10% aqueous sodium carbonate (100 mL). The precipitate was vacuum filtered off and the filter cake washed with cold water (50 mL) and diethylether (10 mL) and dried in the vacuum oven. The crude product was dissolved in acetonitrile (50 mL) and sodium 2-chloro-2,2-difluoroacetate (3.89 g, 25.53 mmol) was added. The mixture was heated to reflux overnight. After cooling to r.t., sat. aqueous ammonium chloride (50 mL) was added and the mixture extracted with ethyl acetate (3×50 mL). The combined organic fractions were washed with water (50 mL), dried with magnesium sulfate and concentrated in vacuo. The product was purified by gradient column chromatography (40 g silica column, 0-20% ethyl acetate in heptane) to give the title compound (1.10 g, 8% yield):

¹H NMR (500 MHz, DMSO-d₆) δ ppm 7.67 (s, 1H), 7.44 (s, 1H), 7.26 (s, 1H), 2.42 (s, 3H); MS (CI+) m/z 237, 239 [M]⁺.

### Example 18i

### Methyl 3-(2-(1,3-dioxolan-2-yl)ethoxy)benzoate

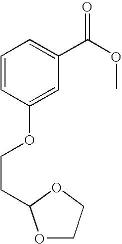

Sodium hydride, 60% dispersion in mineral oil (1.577 g, 39.44 mmol) was slurried in anhydrous N,N-dimethylformamide (100 mL) under argon. The reaction mixture was cooled to 0° C. and methyl 3-hydroxybenzoate (5.00 g, 32.86 mmol) was added in portions over 30 minutes. The solution was stirred at 0° C. for 20 minutes, then at ambient temperature for 1 h. The reaction was cooled to 10° C. during the dropwise addition of 2-(2-bromoethyl)-1,3-dioxolane (4.63 mL, 39.44 mmol). The temperature was raised to 21° C. and the reaction was stirred for 1.5 h, then it was left at ambient temperature for 21 hours. The reaction was cooled to 0° C., quenched with crushed ice and partitioned between dichloromethane (200 mL) and saturated aqueous NaHCO₃ (100 mL). The aqueous layer was extracted with dichloromethane and EtOAc. The organics were combined, washed with water, dried (Na₂SO₄), filtered and concentrated until mainly N,N-dimethylformamide was left with the product. The solution was partitioned between water and diethylether (×2). The organics were combined, dried (Na₂SO₄) and evaporated to give a crude product (9.6 g, quantitative yield) that was used as such in the next step:

¹H NMR (400 MHz, aceton-d₆) δ ppm 7.59 (dt, 1H) 7.52 (dd, 1H) 7.42 (t, 1H) 7.20 (ddd, 1H) 5.05 (t, 1H) 4.18 (t, 2H) 3.93-3.99 (m, 2H) 3.88 (s, 3H) 3.81-3.86 (m, 2H) 2.10 (td, 2H).

### Example 19i

### Methyl 3-(3-oxopropoxy)benzoate

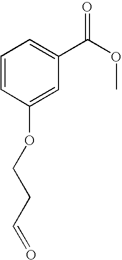

A mixture of water (100 mL) and acetic acid (100 mL) was added to methyl 3-(2-(1,3-dioxolan-2-yl)ethoxy)benzoate (9.6 g, 38.06 mmol). The flask was sealed and heated at 60° C. for 330 minutes. The reaction was cooled to 0° C. and a cold solution of NaOH (65 g) in water (200 mL) was added dropwise to give pH ˜7. The neutral aqueous solution was extracted with EtOAc (×2), and the organics were combined, dried Na₂SO₄, filtered and evaporated to give the crude product (7.20 g, 91% yield) which was used as such in the next step:

¹H NMR (400 MHz, DMSO-d₆) δ ppm 9.69-9.76 (m, 1H) 7.53-7.57 (m, 1H) 7.41-7.46 (m, 2H) 7.18-7.25 (m, 1H) 4.33 (t, 2H) 3.84 (s, 3H) 2.89 (td, 2H).

### Example 20i

### Methyl 3-(3,3-difluoropropoxy)benzoate

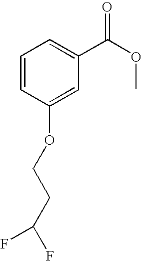

To a solution of methyl 3-(3-oxopropoxy)benzoate (18.00 mg, 0.09 mmol) in dry dichloromethane (0.7 mL) was added diethylaminosulfur trifluoride (10.59 μL, 0.09 mmol). The atmosphere was changed to argon, the vial was sealed and heated with microwaves at 70° C. for 15 minutes. The reaction was diluted with EtOAc (1.5 mL) and washed with saturated NaHCO₃ (1.0 mL). The water phase was extracted with EtOAc (1.0 mL). The organics were combined, dried (Na₂SO₄), filtered and evaporated to give the crude product (18 mg, 90% yield), which was used as such in next step:

¹H NMR (400 MHz, Aceton-d₆) δ ppm 7.61 (dt, 1H) 7.54 (dd, 1H) 7.44 (t, 1H) 7.23 (ddd, 1H) 6.08-6.41 (tt, 1H) 4.25 (t, 2H) 3.88 (s, 3H) 2.31-2.47 (m, 2H); MS (CI) m/z 231 [M+1]⁺.

### Example 21i

### 3-(3,3-Difluoropropoxy)benzoic acid

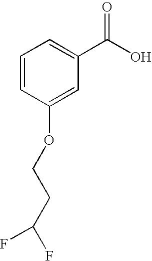

Methyl 3-(3,3-difluoropropoxy)benzoate (2.360 g, 10.25 mmol) was dissolved in tetrahydrofuran (50 mL) and a solution of lithium hydroxide monohydrate (0.570 mL, 20.50 mmol) in water (25.00 mL) was added. The mixture was heated at 50° C. under an atmosphere of argon for 20 h. The mixture was allowed to cool to room temperature and was partitioned between EtOAc (×2) and saturated aqueous NaHCO₃. The combined organic layers were extracted with saturated aqueous NaHCO₃ (×2). The aqueous layers were combined and then acidified (6 M HCl, pH ˜1) and then extracted with dichloromethane (×2). The organics were combined, dried (Na₂SO₄), filtered and evaporated to give the title compound (2.12 g, 96% yield):

¹H NMR (400 MHz, DMSO-d₆) δ ppm 13.03 (br. s., 1H) 7.53 (ddd, 1H) 7.37-7.47 (m, 2H) 7.20 (ddd, 1H) 6.26 (tt, 1H) 4.16 (t, 2H) 2.23-2.40 (m, 2H); MS (ES−) m/z 215 [M−1]⁻.

### Example 22i

### 3-(3,3-Difluoropropoxy)benzoyl chloride

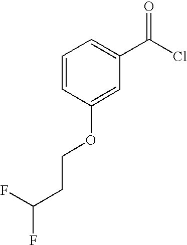

To a solution of 3-(3,3-difluoropropoxy)benzoic acid (1.000 g, 4.63 mmol) in dichloromethane (10 mL) and DMF (0.05 mL) was oxalyl chloride (0.404 mL, 4.63 mmol) added dropwise over 2 min. The solution was stirred at ambient temperature for 2 h, then concentrated in vacuo. The residue was coevaporated with toluene repeatedly to give 3-(3,3-difluoropropoxy)benzoyl chloride (1.080 g, 100% yield). The compound was used as such in next step:

MS (CI) m/z 231 (MeOH quenched) [M+1]⁺.

### Example 23i

### (2-Cyanopyridin-3-yl)zinc(II) bromide

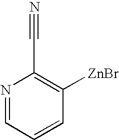

3-Bromopicolinonitrile (11.66 g, 63.71 mmol) was dissolved in dry THF (30 mL) and added dropwise over 1 h to a bottle of Rieke® Zinc in THF (100 mL, 152.91 mmol) under argon. The mixture was stirred 1 h at room temperature and then left at −20° C. for 36 h. The excess zinc was separated off by decantation. The title compound was used as such in the next step:

MS (CI) m/z 105 (H₂O quenched) [M+1]⁺.

### Example 24i

### 3-(3-(3,3-Difluoropropoxy)benzoyl)picolinonitrile

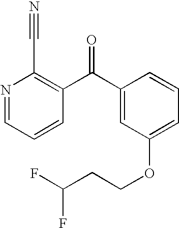

3-(3,3-Difluoropropoxy)benzoyl chloride (1.08 g, 4.60 mmol) and tetrakis(triphenylphosphine)palladium(0) (0.053 g, 0.05 mmol) were mixed in tetrahydrofuran (10 mL) under argon atmosphere at room temperature. (2-Cyanopyridin-3-yl)zinc(II) bromide (0.49 M in THF) (9.39 mL, 4.60 mmol) was added over 2 minutes and the reaction was stirred at ambient temperature over night. Additional tetrakis(triphenylphosphine)palladium(0) (0.479 g, 0.41 mmol) was added and the resulting mixture was stirred for three days. Additional tetrakis(triphenylphosphine)palladium(0) (0.479 g, 0.41 mmol) was added and the resulting mixture was stirred for two days. The reaction was quenched with water (25 mL). NaHCO₃ was added and the product was extracted with dichloromethane (×2). The organics were combined, dried (Na₂SO₄), filtered and evaporated to give the crude product. Purification by silica chromatography using 0 to 50% ethyl acetate in heptane gave 3-(3-(3,3-difluoropropoxy)benzoyl)picolinonitrile (0.572 g, 41% yield):

¹H NMR (500 MHz, CDCl₃) δ ppm 8.84-8.92 (m, 1H) 7.94-8.01 (m, 1H) 7.63-7.68 (m, 1H) 7.41-7.47 (m, 2H) 7.29 (d, 1H) 7.23 (dd, 1H) 5.95-6.24 (m, 1H) 4.17-4.25 (m, 2H) 2.31-2.44 (m, 2H); MS (CI) m/z 303 [M+1]⁺.

### Example 25i

### N-((2-Cyanopyridin-3-yl)(3-(3,3-difluoropropoxy)phenyl)methylene)-2-methylpropane-2-sulfinamide

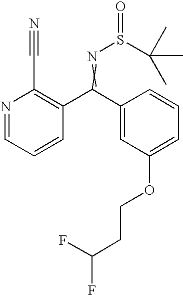

Titanium(IV) ethoxide (0.989 mL, 4.73 mmol) was added to a solution of 3-(3-(3,3-difluoropropoxy)benzoyl)picolinonitrile (572.1 mg, 1.89 mmol) in THF (10 mL) at room temperature under an argon atmosphere. The mixture was stirred for 5 min, then 2-methylpropane-2-sulfinamide (298 mg, 2.46 mmol) was added and the resulting mixture was refluxed for 24 h, then stirred at 50° C. for 2 days. The reaction mixture was cooled to room temperature, then methanol (2 mL), aqueous sodium bicarbonate (sat.) (2 mL) and ethyl acetate (5 mL) was added. The precipitate was filtered off through a pad of Na₂SO₄ on top of celite, and rinsed with ethyl acetate repeatedly. The filtrate was concentrated in vacuo. Purification by silica chromatography using 0 to 50% ethyl acetate in heptane gave N-((2-cyanopyridin-3-yl)(3-(3,3-difluoroprop oxy)phenyl)methylene)-2-methylpropane-2-sulfinamide (463 mg, 60% yield):

¹H NMR (400 MHz, DMSO-d₆) δ ppm 8.83 (d, 1H) 8.01-8.23 (m, 1H) 7.84 (d, 1H) 7.45 (t, 1H) 7.26 (dd, 1H) 7.09-7.14 (m, 1H) 7.06 (d, 1H) 6.26 (tt, 1H) 4.11-4.20 (m, 2H) 2.24-2.40 (m, 2H) 1.28 (br. s., 9H); MS (ES+) m/z 406 [M+1]⁺.

### Example 26i

### 4-Bromo-2-(difluoromethoxy)pyridine

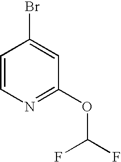

4-Bromopyridin-2(1H)-one (200 mg, 1.15 mmol) and sodium 2-chloro-2,2-difluoroacetate (210 mg, 1.38 mmol) were slurried in dry acetonitrile (8 mL). The mixture was refluxed overnight, allowed to cool to r.t. and directly extracted with pentane (3×5 mL). The combined organic phases were evaporated to give 219 mg (85% yield) of the title compound:

¹H-NMR (500 MHz DMSO-d₆) δ 8.18 (d, 1H), 7.70 (t, 1H), 7.56 (d, 1H), 7.50 (s, 1H); MS (CI+) m/z 226 224 [M+1]⁺

### Example 1

### 5-(3-Isobutoxyphenyl)-5-(pyridin-4-yl)-5H-pyrrolo[3,4-b]pyridin-7-amine

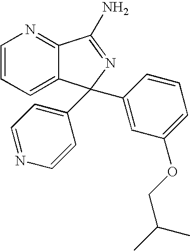

Diisopropyl azodicarboxylate (0.073 mL, 0.37 mmol) was added to a mixture of tert-butyl 5-(3-hydroxyphenyl)-5-(pyridin-4-yl)-5H-pyrrolo[3,4-b]pyridin-7-ylcarbamate (100 mg, 0.25 mmol), triphenylphosphine (98 mg, 0.37 mmol) and 2-methyl-1-propanol (0.034 mL, 0.37 mmol) in THF (0.25 mL).The mixture was ultrasonicated for 45 min and stirred for 3 h at r.t. EtOAc was added and the mixture was washed with NH₄OH (conc.), and brine, dried over MgSO₄ and concentrated. The residue was dissolved in ethyl acetate (5 mL) and hydrogen chloride in water (5.59 mL, 33.54 mmol) was added. The resulting mixture was stirred over night. An additional portion of HCl (6M) was added and the mixture was washed with DCM. NH₄OH (conc.) was added to the water phase and it was extracted with DCM. The organic phase was dried over MgSO₄ and concentrated. The residue was dissolved in MeOH and purified using preparative HPLC. The fractions were concentrated and freeze dried to give the title compound. (0.034 g, 38% yield):

¹H NMR (500 MHz, DMSO-d₆) δ ppm 8.64-8.66 (m, 1H), 8.43-8.47 (m, 2H), 8.29-8.33 (m, 1H), 7.46-7.50 (m, 1H), 7.28-7.32 (m, 2H), 7.19 (t, 1H), 6.83-6.94 (m, 4H), 0.93 (d, 6H), 6.78-6.83 (m, 1H), 3.64 (d, 2H), 1.91-1.98 (m, 1H), 1.90 (s, acetate). MS (ES+) m/z 359 [M+1]⁺.

### Example 2

### 5-(3-(Isopentyloxy)phenyl)-5-(pyridin-4-yl)-5H-pyrrolo[3,4-b]pyridin-7-amine

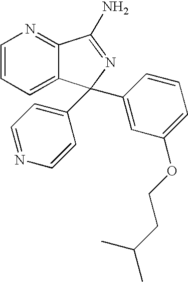

The title compound was synthesized as described for Example 1 in 38% yield starting from tert-butyl 5-(3-hydroxyphenyl)-5-(pyridin-4-yl)-5H-pyrrolo[3,4-b]pyridin-7-ylcarbamate (120 mg, 0.30 mmol) and 3-methyl-1-butanol (0.039 mL, 0.36 mmol):

¹H NMR (500 MHz, DMSO-d₆) δ ppm 8.65 (d, 1H), 8.45 (d, 2H), 8.31 (d, 1H), 7.44-7.51 (m, 1H), 7.27-7.33 (m, 2H), 7.16-7.22 (m, 1H), 6.83-7.05 (m, 3H), 6.76-6.83 (m, 2H), 3.89 (t, 2H), 1.91 (s, acetate), 1.66-1.76 (m, 1H), 1.49-1.58 (m, 2H), 0.88 (d, 6H); MS (ES) m/z 373 [M+1]⁺.

### Example 3

### 5-(3-(Cyclopentylmethoxy)phenyl)-5-(pyridin-4-yl)-5H-pyrrolo[3,4-b]pyridin-7-amine

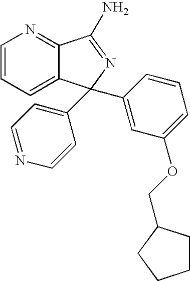

The title compound was synthesized as described for Example 1 in 23% yield starting from tert-butyl 5-(3-hydroxyphenyl)-5-(pyridin-4-yl)-5H-pyrrolo[3,4-b]pyridin-7-ylcarbamate (125 mg, 0.31 mmol) and cyclopentanemethanol (0.040 mL, 0.37 mmol).

¹H NMR (500 MHz, DMSO-d₆) δ ppm 8.63-8.67 (m, 1H), 8.40-8.49 (m, 2H), 8.29-8.34 (m, 1H), 7.46-7.51 (m, 1H), 7.27-7.33 (m, 2H), 7.19 (t, 1H), 6.84-7.00 (m, 3H), 6.78-6.84 (m, 2H), 3.74 (d, 2H), 2.17-2.27 (m, 1H), 1.90 (s, acetate), 1.68-1.76 (m, 2H), 1.46-1.61 (m, 4H), 1.23-1.31 (m, 2H); MS (ES+) m/z 383 [M−1]⁻.

### Example 4

### 5-(3-(Cyclobutylmethoxy)phenyl)-5-(pyridin-4-yl)-5H-pyrrolo[3,4-b]pyridin-7-amine

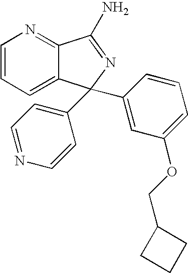

The title compound was synthesized as described for Example 1 in 32% yield starting from tert-butyl 5-(3-hydroxyphenyl)-5-(pyridin-4-yl)-5H-pyrrolo[3,4-b]pyridin-7-ylcarbamate (125 mg, 0.31 mmol) and cyclobutanemethanol (0.035 mL, 0.37 mmol).

¹H NMR (500 MHz, DMSO-d₆) δ ppm 8.63-8.67 (m, 1H), 8.43-8.47 (m, 2H), 8.29-8.33 (m, 1H), 7.46-7.50 (m, 1H), 7.28-7.33 (m, 2H), 7.19 (t, 1H), 6.84-7.00 (m, 3H), 6.79-6.84 (m, 2H), 3.84 (d, 2H), 2.60-2.69 (m, 1H), 1.98-2.06 (m, 2H), 1.73-1.90 (m, 4H), 1.90 (s, acetate); MS (ES−) m/z 369 [M−b 1]⁻.

### Example 5

### 5-(3-((2,2-Difluorocyclopropyl)methoxy)phenyl)-5-(pyridin-4-yl)-5H-pyrrolo[3,4-b]pyridin-7-amine

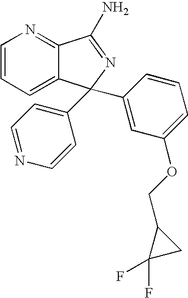

The title compound was synthesized as described for Example 1 in 25% yield starting from tert-butyl 5-(3-hydroxyphenyl)-5-(pyridin-4-yl)-5H-pyrrolo[3,4-b]pyridin-7-ylcarbamate (125 mg, 0.31 mmol) and 2,2-difluorocyclopropylmethanol (40.3 mg, 0.37 mmol):

¹H NMR (500 MHz, DMSO-d₆) δ ppm 8.63-8.67 (m, 1H), 8.43-8.47 (m, 2H), 8.31-8.35 (m, 1H), 7.46-7.51 (m, 1H), 7.29-7.33 (m, 2H), 7.21 (t, 1H), 6.87-7.01 (m, 3H), 6.82-6.87 (m, 2H), 4.01-4.09 (m, 1H), 3.85-3.92 (m, 1H), 2.09-2.21 (m, 1H), 1.90 (s, acetate), 1.63-1.73 (m, 1H), 1.41-1.49 (m, 1H); MS (ES−) m/z 391 [M−1]⁻.

### Example 6

### 5-(3-(3-Fluoropropoxy)phenyl)-5-(2-(trifluoromethyl)pyridin-4-yl)-5H-pyrrolo[3,4-b]pyridin-7-amine

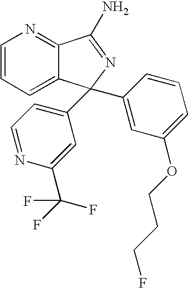

The title compound was synthesized as described for Example 1 in 28% yield starting from tert-butyl 5-(3-hydroxyphenyl)-5-(2-(trifluoromethyl)pyridin-4-yl)-5H-pyrrolo[3,4-b]pyridin-7-ylcarbamate (110 mg, 0.23 mmol) and 3-fluoropropan-1-ol (0.023 mL, 0.30 mmol):

¹H NMR (500 MHz, DMSO-d₆) δ ppm 8.65-8.70 (m, 2H), 8.41-8.45 (m, 1H), 7.71-7.74 (m, 1H), 7.66-7.70 (m, 1H), 7.50-7.54 (m, 1H), 7.20-7.26 (m, 1H), 7.05 (br. s., 2H), 6.90-6.95 (m, 1H), 6.84-6.88 (m, 2H), 4.61 (t, 1H), 4.51 (t, 1H), 3.98 (t, 2H), 1.99-2.10 (m, 2H), 1.88 (s, acetate); MS (ES+) m/z 431 [M+1]⁺.

### Example 7

### 5-(3-(Cyclobutylmethoxy)phenyl)-5-(2-(trifluoromethyl)pyridin-4-yl)-5H-pyrrolo[3,4-b]pyridin-7-amine

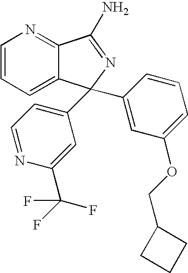

The title compound was synthesized as described for Example 1 in 12% yield starting from tert-butyl 5-(3-hydroxyphenyl)-5-(2-(trifluoromethyl)pyridin-4-yl)-5H-pyrrolo[3,4-b]pyridin-7-ylcarbamate (110 mg, 0.23 mmol) and cyclobutanemethanol (0.029 mL, 0.30 mmol):

¹H NMR (500 MHz, DMSO-d₆) δ ppm 8.68 (d, 2H), 8.39-8.47 (m, 1H), 7.70-7.75 (m, 1H), 7.66-7.70 (m, 1H), 7.49-7.55 (m, 1H), 7.18-7.25 (m, 1H), 7.05 (br. s., 2H), 6.87-6.93 (m, 1H), 6.80-6.87 (m, 2H), 3.85 (d, 2H), 2.58-2.68 (m, 1H), 1.97-2.05 (m, 2H), 1.91 (s, acetate), 1.72-1.90 (m, 4H); MS (ES+) m/z 439 [M+1]⁺.

### Example 8

### 5-(3-(Cyclopentyloxy)phenyl)-5-(pyridin-4-yl)-5H-pyrrolo[3,4-b]pyridin-7-amine

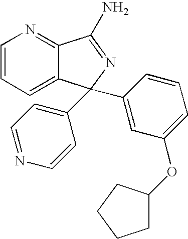

Cyclopentyl bromide (0.021 mL, 0.19 mmol) was added to a mixture of tert-butyl 5-(3-hydroxyphenyl)-5-(pyridin-4-yl)-5H-pyrrolo[3,4-b]pyridin-7-ylcarbamate (78 mg, 0.19 mmol) and cesium carbonate (63.1 mg, 0.19 mmol) in DMF (1 mL). The reaction mixture was heated to 70° C. for 30 min and then to 120° C. for 20 min. The mixture was filtered and purified using preparative HPLC to give 0.022 g (32% yield) of the title compound.

¹H NMR (500 MHz, DMSO-d₆) δ ppm 8.62-8.67 (m, 1H), 8.43-8.48 (m, 2H), 8.28-8.32 (m, 1H), 7.46-7.50 (m, 1H), 7.29-7.34 (m, 2H), 7.15-7.20 (m, 1H), 6.80-7.01 (m, 3H), 6.75-6.79 (m, 2H), 4.65-4.72 (m, 1H), 1.88 (s, acetate), 1.78-1.86 (m, 2H), 1.58-1.69 (m, 4H), 1.48-1.58 (m, 2H); MS (ES) m/z 371 [M+1]⁺

### Example 9

### 5-(3-Cyclobutoxyphenyl)-5-(pyridin-4-yl)-5H-pyrrolo[3,4-b]pyridin-7-amine

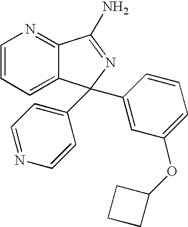

The title compound was synthesized as described for Example 8 in 19% yield starting from tert-butyl 5-(3-hydroxyphenyl)-5-(pyridin-4-yl)-5H-pyrrolo[3,4-b]pyridin-7-ylcarbamate (43 mg, 0.11 mmol) and cyclobutyl bromide (10.06 μL, 0.11 mmol):

¹H NMR (500 MHz, DMSO-d₆) δ ppm 8.63-8.68 (m, 1H), 8.44-8.49 (m, 2H), 8.29-8.34 (m, 1H), 7.46-7.52 (m, 1H), 7.30-7.36 (m, 2H), 7.18 (t, 1H), 6.83-7.01 (m, 3H), 6.67-6.76 (m, 2H), 4.58 (m, 1H), 2.27-2.36 (m, 2H), 1.92-2.02 (m, 2H), 1.90 (s, acetate), 1.70-1.78 (m, 1H) 1.55-1.65 (m, 1H); MS (ES) m/z 357 [M+1]⁺.

### Example 10

### 5-(3-(3-Fluoropropoxy)phenyl)-5-(pyridin-4-yl)-5H-pyrrolo[3,4-b]pyridin-7-amine

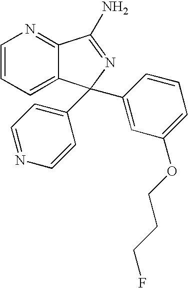

The title compound was synthesized as described for Example 91 in 7% yield starting from N-((2-cyanopyridin-3-yl)(3-(3-fluoropropoxy)phenyl)methylene)-2-methylpropane-2-sulfinamide (340 mg, 0.88 mmol) and 4-iodopyridine (234 mg, 1.14 mmol):

¹H NMR (500 MHz, DMSO-d₆) δ ppm 8.63-8.67 (m, 1H), 8.42-8.47 (m, 2H), 8.29-8.34 (m, 1H), 7.45-7.50 (m, 1H), 7.28-7.33 (m, 2H), 7.21 (t, 1H), 6.87-6.98 (m, 3H), 6.80-6.87 (m, 2H), 4.61 (t, 1H), 4.51 (t, 1H), 3.97 (t, 2H) 1.99-2.11 (m, 2H); MS (ES) m/z 363 [M+1]⁺.

### Example 11

### 5-(3-(3-Fluoropropoxy)phenyl)-5-(4-methoxy-3,5-dimethylphenyl)-5H-pyrrolo[3,4-b]pyridin-7-amine

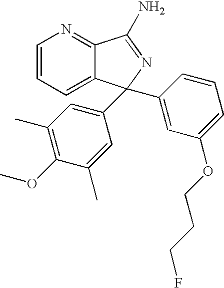

n-Butyllithium (0.264 mL, 0.66 mmol) was added to isopropylmagnesium bromide (0.330 mL, 0.33 mmol) in THF (3 mL) under a nitrogen atmosphere at 0° C. The reaction mixture was stirred 10 min and then cooled to −78° C. 5-Bromo-2-methoxy-1,3-dimethylbenzene (133 mg, 0.62 mmol) in THF (1.5 mL) was added dropwise over 10 min and the mixture was stirred for 20 min at −78° C. N-((2-Cyanopyridin-3-yl)(3-(3-fluoropropoxy)phenyl)methylene)-2-methylpropane-2-sulfinamide (160 mg, 0.41 mmol) in THF (1.5 mL) was added and stirring was continued for 1.5 h at −78° C., and then the temperature was allowed to reach r.t. Water and NaHCO₃ (sat.) was added and the mixture was extracted with EtOAc. The organic phase was dried over MgSO₄ and concentrated. The residue was dissolved in methanol (8 mL) and hydrogen chloride (1M in diethyl ether) (0.826 mL, 0.83 mmol) was added, and the mixture was stirred over night. DCM, water and NH₄OH (conc.) was added until the pH reached-9-10. The organic phase was collected and concentrated and the residue was purified with preparative HPLC to give 0.056 g (32% yield).

¹H NMR (500 MHz, DMSO-d₆) δ ppm 8.58-8.62 (m, 1H), 8.19-8.23 (m, 1H), 7.41-7.46 (m, 1H), 7.17 (t, 1H), 6.97 (s, 2H), 6.87-6.90 (m, 1H), 6.82-6.84 (m, 1H), 6.77-6.81 (m, 1H), 6.71 (br. s., 2H), 4.61 (t, 1H), 4.51 (t, 1H), 3.96 (t, 2H), 3.58 (s, 3H), 2.13 (s, 6H), 1.99-2.10 (m, 2H), 1.90 (s, acetate); MS (ES+) m/z 420 [M+1]⁺

### Example 12

### 5-(3-(3-Fluoropropoxy)phenyl)-5-(2-methoxypyridin-4-yl)-5H-pyrrolo[3,4-b]pyridin-7-amine

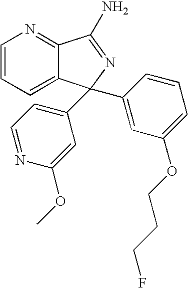

The title compound was synthesized as described for Example 9i. in 13% yield starting from N-((2-cyanopyridin-3-yl)(3-(3-fluoroprop oxy)phenyl)methylene)-2-methylpropane-2-sulfinamide (120 mg, 0.31 mmol) and 4-iodo-2-methoxypyridine (95 mg, 0.40 mmol): ¹H NMR (500 MHz, DMSO-d₆) δ ppm 8.63-8.66 (m, 1H), 8.29-8.32 (m, 1H), 8.02-8.05 (m, 1H), 7.45-7.49 (m, 1H), 7.20 (t, 1H), 6.87-7.00 (m, 3H), 6.81-6.87 (m, 3H), 6.65-6.67 (m, 1H), 4.61 (t, 1H), 4.52 (t, 1H), 3.97 (t, 2H), 3.78 (s, 3H), 1.99-2.10 (m, 2H); MS (ES) m/z 391 [M−1]⁻.

### Example 13

### 5-(3-(3-Fluoropropoxy)phenyl)-5-(2-methylpyridin-4-yl)-5H-pyrrolo[3,4-b]pyridin-7-amine

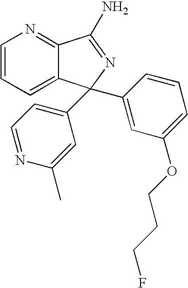

The title compound was synthesized as described for Example 11 in 23% yield starting from N-((2-cyanopyridin-3-yl)(3-(3-fluoropropoxy)phenyl)methylene)-2-methylpropane-2-sulfinamide (120 mg, 0.31 mmol) and 4-bromo-2-methylpyridine (80 mg, 0.46 mmol).

¹H NMR (500 MHz, DMSO-d₆) δ ppm 8.62-8.67 (m, 1H), 8.28-8.34 (m, 2H), 7.45-7.50 (m, 1H), 7.16-7.23 (m, 2H), 7.10-7.13 (m, 1H), 7.10-7.13 (m, 1H), 6.81-6.95 (m, 5H), 4.61 (t, 1H), 4.51 (t, 1H), 3.96 (t, 2H), 2.39 (s, 3H), 1.90 (s, acetate) 1.99-2.10 (m, 2H); MS (ES+) m/z 377 [M+1]⁺.

### Example 14

### 5-(3-(3-Fluoropropoxy)phenyl)-5-(4-methoxy-3-(trifluoromethyl)phenyl)-5H-pyrrolo[3,4-b]pyridin-7-amine

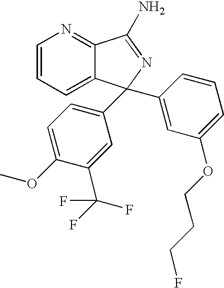

The title compound was synthesized as described for Example 91 in 5% yield starting from N-((2-cyanopyridin-3-yl)(3-(3-fluoropropoxy)phenyl)methylene)-2-methylpropane-2-sulfinamide (120 mg, 0.31 mmol) and 4-bromo-1-methoxy-2-(trifluoromethyl)benzene (111 mg, 0.43 mmol):

¹H NMR (600 MHz, DMSO-d₆) δ ppm 8.61-8.65 (m, 1H), 8.24-8.28 (m, 1H), 7.55-7.58 (m, 1H), 7.52-7.54 (m, 1H), 7.45-7.49 (m, 1H), 7.15-7.22 (m, 2H), 6.86-6.90 (m, 1H), 6.79-6.84 (m, 4H), 4.60 (t, 1H), 4.52 (t, 1H), 3.97 (t, 2H), 3.84 (s, 3H), 2.00-2.09 (m, 2H); MS (ES−) m/z 458 [M−1]⁻.

### Example 15

### 5-(2,6-Dimethylpyridin-4-yl)-5-(3-isobutoxyphenyl)-5H-pyrrolo[3,4-b]pyridin-7-amine acetate

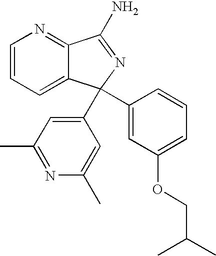

The title compound was synthesized as described for Example 1 in 37% yield starting from N-((2-cyanopyridin-3-yl)(3-isobutoxyphenyl)methylene)-2-methylpropane-2-sulfinamide (96 mg, 0.25 mmol) and 4-bromo-2,6-dimethylpyridine (60.5 mg, 0.33 mmol):

¹H-NMR (500 MHz, DMSO-d₆) δ ppm 8.64 (d, 1H), 8.29 (d, 1H), 7.47 (dd, 1H), 7.18 (t, 1H), 6.97 (s, 2H), 6.87-6.77 (m, 5H), 3.64 (d, 2H), 2.35 (s, 6H), 1.95 (m, 1H), 0.93 (d, 6H); MS (ES+) m/z 387 [M+1]⁺

### Example 16

### 5-(3-Isobutoxyphenyl)-5-(6-methoxy-5-methylpyridin-3-yl)-5H-pyrrolo[3,4-b]pyridin-7-amine acetate

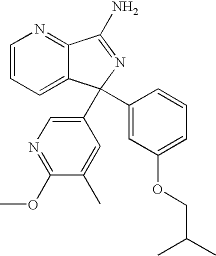

The title compound was synthesized as described for Example 91. in 12% yield starting from N42-cyanopyridin-3-yl)(3-isobutoxyphenyl)methylene)-2-methylpropane-2-sulfinamide (92 mg, 0.24 mmol) and 5-bromo-2-methoxy-3-methylpyridine (48.5 mg, 0.24 mmol), 0.24 mmol):

¹H-NMR (500 MHz, DMSO-d₆) δ ppm 8.62 (d, 1H), 8.29 (d, 1H), 7.89 (d, 1H), 7.48-7.44 (m, 2H), 7.16 (t, 1H), 6.87 (d, 1H), 6.84-6.73 (m, 4H), 3.81 (s, 3H), 3.64 (d, 2H), 2.07 (s, 3H), 1.95 (m, 1H), 0.94 (d, 6H); MS (ES) m/z 403 [M+1]⁺

### Example 17

### 5-(3-Isobutoxyphenyl)-5-(6-methoxy-5-methylpyridin-3-yl)-5H-pyrrolo[3,4-b]pyridin-7-amine acetate

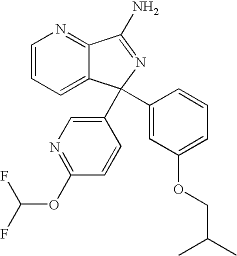

The title compound was synthesized as described for Example 91 in 3% yield starting from N-((2-cyanopyridin-3-yl)(3-isobutoxyphenyl)methylene)-2-methylpropane-2-sulfinamide (92 mg, 0.24 mmol) and 5-bromo-2-(difluoromethoxy)pyridine (99 mg, 0.44 mmol)

¹H-NMR (500 MHz, CDCl₃) δ ppm 8.66 (d, 1H), 8.07 (d, 1H), 7.91 (dd, 1H), 7.73 (dd, 1H), 7.42-7.39 (m, 1H), 7.41 (t, 1H), 7.21 (t, 1H), 6.87-6.79 (m, 4H), 3.66 (d, 2H), 2.03 (m, 1H), 1.00 (d, 6H); MS (ES) m/z 425 [M+1]⁺

### Example 18

### 5-(4-(Difluoromethoxy)phenyl)-5-(3-isobutoxyphenyl)-5H-pyrrolo[3,4-b]pyridin-7-amine

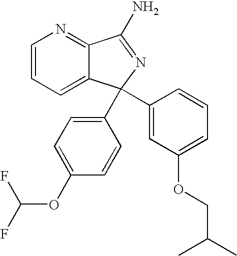

The title compound was synthesized as described for Example 1 in 21% yield starting from N-((2-cyanopyridin-3-yl)(3-isobutoxyphenyl)methylene)-2-methylpropane-2-sulfinamide (110 mg, 0.29 mmol) and 1-bromo-4-(difluoromethoxy)benzene (77 mg, 0.34 mmol):

¹H NMR (500 MHz, DMSO-d₆) δ 6 ppm 0.94 (d, 6H) 1.91 (s, acetate) 1.92-1.99 (m, 1H) 3.64 (d, 2H) 6.68-6.91 (m, 5H) 7.02-7.10 (m, 2H) 7.15-7.21 (m, 1H) 7.33-7.38 (m, 2H) 7.45-7.49 (m, 1H) 8.21-8.26 (m, 1H) 8.62-8.65 (m, 1H). MS (ES) m/z 424 [M+1]⁺

### Example 19

### 5-(2-(Difluoromethoxy)-6-methylpyridin-4-yl)-5-(3-(3-fluoropropoxy)phenyl)-5H-pyrrolo[3,4-b]pyridin-7-amine

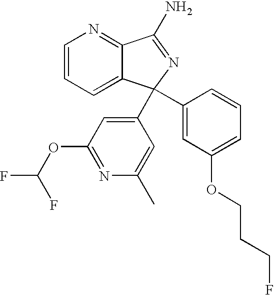

4-Bromo-2-difluoromethoxy-6-methylpyridine (228 mg, 0.96 mmol) was dissolved in dry tetrahydrofurane (2 mL) under argon atmosphere and the resulting solution was cooled to −69° C. (external thermometer). 1.7 M tert-butyl lithium (1.129 mL, 1.92 mmol) was added dropwise over 1 min. After 10 min, a solution of N-((2-cyanopyridin-3-yl)(3-(3-fluoropropoxy)phenyl)methylene)-2-methylpropane-2-sulfinamide (286 mg, 0.74 mmol) in dry tetrahydrofurane (2 mL) was added dropwise over 3 min. The resulting solution was stirred another 30 min at the indicated temperature, and then brought to r.t. over 1 h. 1.25 M hydrogen chloride in methanol (2 mL) was added and the solution stirred for 1 h at rt. The solvents were evaporated and the remaining residue was partitioned between chloroform (5 mL) and 10% aq. sodium carbonate (5 mL). The organic phase was separated and concentrated in vacuo. The crude product was purified by preparative chromatography (Column; XTerra® Prep C8 10 mm OBD™ 19×300 mm, with guard column; XTerra® Prep MS C8 10 mm 19×10 mm Cartridge. A gradient of 30-70% B (100% MeCN) in A (95% 0.1M NH₄OAc in MilliQ water and 5% MeCN) was used as eluent at a flow rate 20 mL/min.) The desired fractions were freeze-dried overnight to give a the product as the acetate salt (13 mg, 4% yield):

¹H NMR (500 MHz, DMSO-d₆) δ ppm 8.55 (d, 1H), 8.39 (d, 1H), 7.62 (t, 1H), 7.48 (m, 1H), 7.21 (t, 1H), 7.10 (s, 1H), 6.95-6.83 (m, 5H), 6.72 (s, 1H), 4.61 (t, 1H), 4.52 (t, 1H), 3.98 (t, 2H), 2.36 (s, 3H), 2.09-2.01 (m, 2H); MS (ES+) m/z 443 [M+1]⁺.

### Example 20

### 5-(3-Chloro-4-methoxyphenyl)-5-(3-(3-fluoropropoxy)phenyl)-5H-pyrrolo[3,4-b]pyridin-7-amine

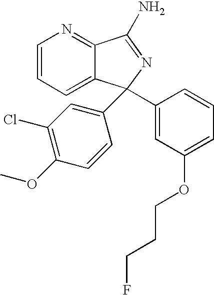

tert-Butyllithium (1.7 M in pentane, 0.607 mL, 1.03 mmol) was added dropwise to THF (2 mL) at −100° C. under an argon atmosphere. Then a solution of 4-bromo-2-chloro-1-methoxybenzene (137 mg, 0.62 mmol) in THF (0.5 mL) was added dropwise followed by the addition of N-((2-cyanopyridin-3-yl)(3-(3-fluoropropoxy)phenyl)methylene)-2-methylpropane-2-sulfinamide (200 mg, 0.52 mmol) in THF (2 mL). The resulting reaction mixture was left on the thawing cooling bath for 30 min and then the cooling bath was removed and the mixture was stirred at rt for 1 h. Hydrogen chloride 1.25 M in methanol (2.478 mL, 3.10 mmol) was added and the resulting mixture was stirred at rt for 1 h. The mixture was concentrated and purified by preparative HPLC. The fractions containing the product were pooled and the MeCN was removed in vacuo. Saturated aqueous NaHCO₃ was added to the residue and the mixture was extracted with DCM (3×10 mL). The combined organics were passed through a phase separator and concentrated to give 75 mg (34% yield) of the title compound:

¹H NMR (DMSO-d₆) δ ppm 8.55-8.69 (m, 1H) 8.19-8.32 (m, 1H) 7.42-7.52 (m, 1H) 7.27-7.32 (m, 1H) 7.22-7.27 (m, 1H) 7.16-7.22 (m, 1H) 7.01-7.07 (m, 1H) 6.86-6.91 (m, 1H) 6.67-6.86 (m, 4H) 4.61 (t, 1H) 4.52 (t, 1H) 3.96 (t, 2H) 3.80 (s, 3H) 2.05-2.10 (m, 1H) 2.00-2.05 (m, 1H); MS (ES+) m/z 426, 428 [M+1]⁺.

### Example 21

### 5-(3-(3,3-Difluoropropoxy)phenyl)-5-(2-(trifluoromethyl)pyridin-4-yl)-5H-pyrrolo[3,4-b]pyridin-7-amine trifluoroacetic acid salt

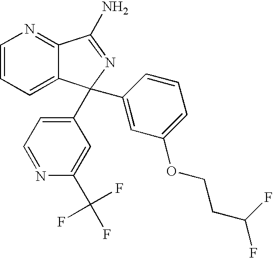

tert-Butyllithium (1.6 M in pentane) (0.617 mL, 0.99 mmol) was dropwise added to dry THF (10.00 mL) under argon at −100° C. 4-Bromo-2-(trifluoromethyl)pyridine (0.111 g, 0.49 mmol) in dry THF (2.000 mL) was added dropwise. The reaction mixture was stirred at −100° C. for 10 min, then N-((2-cyanopyridin-3-yl)(3-(3,3-difluoropropoxy)phenyl)methylene)-2-methylpropane-2-sulfinamide (0.200 g, 0.49 mmol) in dry THF (2.000 mL) was added dropwise. The mixture was stirred at −100° C. for 30 min, then at −70° C. for 2 h, and then hydrochloric acid (0.5 M in methanol) (2.96 mL, 1.48 mmol) was added. The resulting mixture was stirred for 30 min at −70° C., and then it was allowed to reach room temperature. It was stirred for 30 min, and then concentrated in vacuo. The residue was partitioned between aqueous sodium bicarbonate (sat.) and dichloromethane (×3). The combined organic layers were dried (Na₂SO₄), filtered and concentrated in vacuo. The product was purified by prep-HPLC to give 5-(3-(3,3-difluoropropoxy)phenyl)-5-(2-(trifluoromethyl)pyridin-4-yl)-5H-pyrrolo[3,4-b]pyridin-7-amine (0.073 g, 26% yield):

¹H NMR (400 MHz, DMSO-d₆) δ ppm 12.27 (br. s., 1H) 10.44 (br. s., 1H) 10.09 (br. s., 1H) 8.94-9.01 (m, 1H) 8.82 (d, 1H) 8.58 (dd, 1H) 7.91 (dd, 1H) 7.84 (d, 1H) 7.71 (dd, 1H) 7.37 (t, 1H) 7.05 (dd, 1H) 7.10 (t, 1H) 6.84 (dd, 1H) 6.80 (t, 1H) 6.21 (tt, 1H) 4.07 (t, 2H) 2.19-2.35 (m, 2H); MS (ES+) m/z 449 [M+1]⁺.

### Example 22

### 5-(3-(3,3-Difluoropropoxy)phenyl)-5-(5-methoxy-4,6-dimethylpyridin-2-yl)-5H-pyrrolo[3,4-b]pyridin-7-amine acetic acid salt

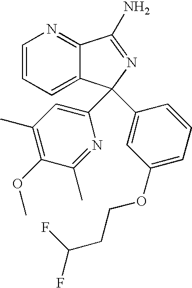

tert-Butyllithium (1.7 M in pentane) (0.850 mL, 1.44 mmol) was added dropwise to THF (5 mL) at −100° C. under an argon atmosphere. A solution of 6-bromo-3-methoxy-2,4-dimethylpyridine (156 mg, 0.72 mmol) in THF (3 mL) was added dropwise followed by the addition of N-((2-cyanopyridin-3-yl)(3-(3,3-difluoropropoxy)phenyl)methylene)-2-methylpropane-2-sulfinamide (244 mg, 0.60 mmol) in THF (7 mL). The resulting reaction mixture was left on the thawing cooling bath for 30 min and then hydrogen chloride (0.5 M in methanol) (7.22 mL, 3.61 mmol) was added and the resulting mixture was allowed to reach room temperature over night. The mixture was concentrated in vacuo. The residue was partitioned between aqueous sodium bicarbonate (sat.) and dichloromethane (×3). The combined organic layers were dried (Na₂SO₄), filtered and concentrated in vacuo. The residue was purified by prep-HPLC to give 5-(3-(3,3-difluoropropoxy)phenyl)-5-(5-methoxy-4,6-dimethylpyridin-2-yl)-5H-pyrrolo[3,4-b]pyridin-7-amine (10.2 mg, 3.4% yield):

¹H NMR (400 MHz, DMSO-d₆) δ ppm 8.57 (dd, 1H) 8.43 (dd, 1H) 7.39-7.47 (m, 2H) iii 7.15 (t, 1H) 7.00 (d, 1H) 6.91-6.96 (m, 1H) 6.68-6.87 (m, 2H) 6.21 (t, 1H) 4.00 (t, 2H) 3.63 (s, 3H) 2.38 (s, 3H) 2.15-2.34 (m, 5H) 1.88 (s, 3H); MS (ES+) m/z 439 [M+1]⁺.

### Example 23

### 5-(2-(Difluoromethoxy)pyridin-4-yl)-5-(3-(3-fluoropropoxy)phenyl)-5H-pyrrolo[3,4-b]pyridin-7-amine 1.67 acetic acid

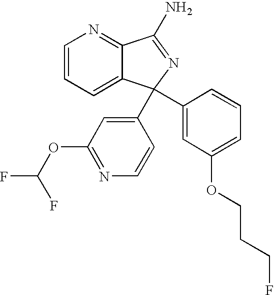

4-Bromo-2-(difluoromethoxy)pyridine (110 mg, 0.49 mmol) was dissolved in dry tetrahydrofurane (2 mL) under argon atmosphere and the resulting solution was cooled to −68° C. (external thermometer). tert-Butyl lithium (1.7 M) (0.578 mL, 0.98 mmol) was added dropwise over 1 min. After 10 min, a solution of N-((2-cyanopyridin-3-yl)(3-(3-fluoropropoxy)phenyl)methylene)-2-methylpropane-2-sulfinamide (286 mg, 0.74 mmol) in dry tetrahydrofurane (2 mL) was added dropwise over 3 min. The resulting solution was stirred another 30 min cold and then brought to r.t. over 1 h. 1.25 M hydrogen chloride in methanol (2 mL) was added and the solution stirred 1 h at rt. The solvents were evaporated and the residue partitioned between chloroform (5 mL) and 10% aqueous sodium carbonate (5 mL). The organic phase was separated and evaporated. The crude product was purified by preparative chromatography (Column; XTerra® Prep C8 10 μm OBD™ 19×300 mm, with guard column; XTerra® Prep MS C8 10 μm 19×10 mm Cartridge. A gradient of 25-65% B (100% MeCN) in A (95% 0.1M NH₄OAc in MilliQ water and 5% MeCN) was used as eluent at flow rate 20 mL/min.) The desired fractions were freeze-dried overnight to give 7 mg (4% yield) of the title compound:

¹H NMR (DMSO-d₆ 500 MHz) δ 8.66 (d, 1H), 8.40 (d, 1H), 8.16 (d, 1H), 7.65 (t, 1H), 7.49 (m, 1H), 7.25-7.20 (m, 2H), 7.0-6.83 (m, 6H), 4.56, (d, 2H), 3.98 (t, 2H), 2.06 (m, 2H); MS (ES+) m/z 429.1 [M+1]⁺.

### Assays

The level of activity of the compounds was tested using the following methods:

### TR-FRET Assay

The β-secretase enzyme used in the TR-FRET is prepared as follows:

The cDNA for the soluble part of the human β-Secretase (AA 1-AA 460) was cloned using the ASP2-Fc10-1-IRES-GFP-neoK mammalian expression vector. The gene was fused to the Fc domain of IgG1 (affinity tag) and stably cloned into HEK 293 cells. Purified sBACE-Fc was stored in −80° C. in Tris buffer, pH 9.2 and had a purity of 95%.

The enzyme (truncated form) was diluted to 6 μg/mL (stock 1.3 mg/mL) and the substrate (Europium)CEVNLDAEFK(Qsy7) to 200 nM (stock 120 μM) in reaction buffer (NaAcetate, chaps, triton x-100, EDTA pH4.5). The robotic systems Biomek FX and Velocity 11 were used for all liquid handling and the enzyme and substrate solutions were kept on ice until they were placed in the robotic system. Enzyme (9 μl) was added to the plate then 1 μl of compound in dimethylsulphoxide was added, mixed and pre-incubated for 10 minutes. Substrate (10 μl) was then added, mixed and the reaction proceeded for 15 minutes at room temperature. The reaction was stopped with the addition of Stop solution (7 μl, NaAcetate, pH 9). The fluorescence of the product was measured on a Victor II plate reader with an excitation wavelength of 340 nm and an emission wavelength of 615 nm. The assay was performed in a Costar 384 well round bottom, low volume, non-binding surface plate (Corning #3676). The final concentration of the enzyme was 2.7 μg/ml; the final concentration of substrate was 100 nM (Km of ˜250 nM). The dimethylsulphoxide control, instead of test compound, defined the 100% activity level and 0% activity was defined by wells lacking enzyme (replaced with reaction buffer). A control inhibitor was also used in dose response assays and had an IC₅₀ of ˜575 nM.

sAPPβ Release Assay

SH-SY5Y cells were cultured in DMEM/F-12 with Glutamax, 10% FCS and 1% non-essential aminoacids and cryopreserved and stored at −140° C. at a concentration of 7.5×10⁶ cells per vial. Thaw cells and seed at a conc. of 1.5×10⁵/ml in DMEM/F-12 with Glutamax, 10% FCS and 1% non-essential aminoacids to a 96-well tissue culture treated plate, 100 μl cell susp/well. The cell plates were then incubated for 7 hours at 37° C., 5% CO2. The cell medium was removed, followed by addition of 90 μl compound diluted in DMEM/F-12 with Glutamax, 10% FCS, 1% non-essential aminoacids and 1% PeSt to a final conc. of 1% DMSO. The compounds were incubated with the cells for 16 h (over night) at 37° C., 5% CO2. Meso Scale Discovery (MSD) plates were used for the detection of sAPPβ release. MSD sAPPβ plates were blocked in 3% BSA in Tris wash buffer (150 μl/well) for 1 hour in RT and washed 4 times in Tris wash buffer (150 μl/well). 50 μl of medium was transferred to the pre-blocked and washed MSD sAPPβ microplates, and the cell plates were further used in an ATP assay to measure cytotoxicity. The MSD plates were incubated with shaking in RT for 1 hour followed by washing 4 times. 25 μl detection antibody was added (1 nM) per well followed by incubation with shaking in RT for 1 h and washing 4 times. 150 μl Read Buffer was added per well and the plates were read in a SECTOR Imager.

### ATP Assay

As indicated in the sAPPβ release assay, after transferring 50 μL medium from the cell plates for sAPPβ detection, the plates were used to analyse cytotoxicity using the ViaLight™ Plus cell proliferation/cytotoxicity kit from Cambrex BioScience that measures total cellular ATP. The assay was performed according to the manufacture's protocol. Briefly, 25 μL cell lysis reagent was added per well. The plates were incubated at room temperature for 10 min. Two min after addition of 50 μL, reconstituted ViaLight™ Plus ATP reagent, the luminescence was measured in a Wallac Victor2 1420 multilabel counter.

### Results

Typical IC₅₀ values for the compounds of the present invention are in the range of about 0.1 to about 30,000 nM. Biological data on exemplified final compounds is given below in Table I.

TABLE I

| Example No. | IC50 in TR-FRET assay |
|---|---|
| 1 | 640 |
| 2 | 1700 |
| 3 | 1500 |
| 4 | 2700 |
| 5 | 2300 |
| 6 | 1500 |
| 7 | 1200 |
| 8 | 3000 |
| 9 | 8000 |
| 10 | 2400 |
| 11 | 81 |
| 12 | 2700 |
| 13 | 2700 |
| 14 | 960 |
| 15 | 3800 |
| 16 | 3100 |
| 17 | 1300 |
| 18 | 150 |
| 19 | 909 |
| 20 | 779 |
| 21 | 1150 |
| 22 | 337 |
| 23 | 2620 |
| 9i | 26000 |

1. A compound according to formula (I) or a pharmaceutically acceptable salt thereof, wherein:
formula (I) corresponds to: 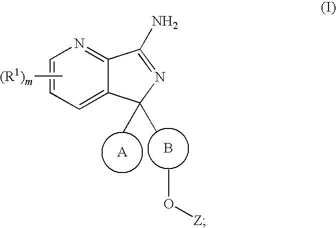
R¹ is selected from halogen, cyano, NO₂, SO₂R², C₁₋₆alkyl, C₂₋₆alkenyl, C₂₋₆alkynyl, NR³R⁴, OR², C(O)R², C(O)NR³R⁴, and COOR², wherein:
the C₁₋₆alkyl, C₂₋₆alkenyl, or C₂₋₆alkynyl is optionally substituted with one or more R⁷;
R² is selected from C₁₋₆alkyl, C₂₋₆alkenyl, and C₂₋₆alkynyl, wherein:
the C₁₋₆alkyl, C₂₋₆alkenyl, or C₂₋₆alkynyl is optionally substituted with one or more R⁷;
as to R³ and R⁴:
R³ and R⁴ are independently selected from hydrogen, C₁₋₆alkyl, C₂₋₆alkenyl, C₂₋₆alkynyl, aryl, heteroaryl, heterocyclyl, and carbocyclyl, wherein:
the C₁₋₆alkyl, C₂₋₆alkenyl, C₂₋₆alkynyl, aryl, heteroaryl, heterocyclyl, or carbocyclyl is optionally substituted with one or more R⁷; or
R³ and R⁴, together with the atom to which they are attached, form a 4 to 7 membered ring;
A is selected from aryl and heteroaryl, wherein:
the aryl or heteroaryl is optionally substituted with one or more R⁵;
B is selected from aryl and heteroaryl, wherein:
the aryl or heteroaryl is optionally substituted with one or more R⁶;
Z is selected from aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl, and C₂₋₆alkynylheterocyclyl, wherein:
the aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl, or C₂₋₆alkynylheterocyclyl is optionally substituted with one to three R⁷;
R⁵ is selected from halo, cyano, C₁₋₆alkyl, C₁₋₆haloalkyl, C₃₋₆cycloalkyl, OC₁₋₆alkyl and OC₁₋₆alkylaryl, wherein:
the C₁₋₆alkyl, C₃₋₆cycloalkyl, OC₁₋₆alkyl, or OC₁₋₆alkylaryl, is optionally substituted with one to three R⁷;
R⁶ is selected from halogen and cyano;
R⁷ is selected from halogen, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, OH, cyano, C(O)OC₁₋₃alkyl and NR⁸R⁹, wherein:
the C₁₋₆alkyl, SO₂C₁₋₃alkyl, OC₁₋₃alkyl, OC₁₋₃haloalkyl, C₁₋₃alkylOH, C₁₋₃alkylNR⁸R⁹, or C(O)OC₁₋₃alkyl is optionally substituted with one or more R¹⁶;
as to R⁸ and R⁹:
R⁸ and R⁹ are independently selected from hydrogen, C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl, and carbocyclyl, wherein:
the C₁₋₆alkyl, C₁₋₆haloalkyl, C₂₋₆alkenyl, C₂₋₆alkynyl, C₁₋₃alkylNR¹¹R¹², C₁₋₃alkylOaryl, heteroaryl, heterocyclyl, or carbocyclyl is optionally substituted with one or more R¹⁰; or
R⁸ and R⁹, together with the atom to which they are attached, form a 4 to 6 membered ring;
R¹⁰ is selected from halo, C₁₋₃alkyl, OC₁₋₃alkyl, and OC₁₋₃haloalkyl;
R¹¹ and R¹² are independently selected from hydrogen, C₁₋₃alkyl, and C₁₋₃haloalkyl; and
m is selected from 0, 1, and 2.

2. A compound or pharmaceutically acceptable salt thereof according to claim 1, wherein:
R¹ is selected from halogen, cyano, NO₂, SO₂R², C₁₋₆alkyl, NR³R⁴, OR², C(O)R², C(O)NR³R⁴, and COOR², wherein:
the C₁₋₆alkyl is optionally substituted with one or more R⁷;
as to R³ and R⁴:
R³ and R⁴ are independently selected from hydrogen, C₁₋₆alkyl, aryl, heteroaryl, heterocyclyl, and carbocyclyl, wherein:
the C₁₋₆alkyl, aryl, heteroaryl, heterocyclyl, or carbocyclyl is optionally substituted with one or more R⁷; or
R³ and R⁴, together with the atom to which they are attached, form a 4 to 7 membered ring;
R⁷ is selected from halogen, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, OH, cyano, and C(O)OC₁₋₃alkyl, wherein:
the C₁₋₆alkyl, SO₂C₁₋₃alkyl, OC₁₋₃alkyl, OC₁₋₃haloalkyl, C₁₋₃alkylOH, C₁₋₃alkylNR⁸R⁹, or C(O)OC₁₋₃alkyl is optionally substituted with one or more R¹⁰; and
as to R⁸ and R⁹:
R⁸ and R⁹ are independently selected from hydrogen, C₁₋₆alkyl, C₁₋₆haloalkyl, C₁₋₃alkylNR¹¹R¹², C₁₋₃alkylOaryl, heteroaryl, heterocyclyl, and carbocyclyl, wherein:
the C₁₋₆alkyl, C₁₋₆haloalkyl, C₁₋₃alkylNR¹¹R¹², C₁₋₃alkylOaryl, heteroaryl, heterocyclyl, or carbocyclyl is optionally substituted with one or more R¹⁰; or
R⁸ and R⁹, together with the atom to which they are attached, form a 4 to 6 membered ring.

3. A compound or pharmaceutically acceptable salt thereof according to claim 1, wherein:
R¹ is selected from halogen, cyano, NO₂, SO₂R², C₁₋₆alkyl, NR³R⁴, OR², and C(O)R², wherein:
the C₁₋₆alkyl is optionally substituted with one or more R⁷;
R² is C₁₋₆alkyl optionally substituted with one or more R⁷;
R³ and R⁴ are independently selected from hydrogen, C₁₋₆alkyl, aryl, heteroaryl, heterocyclyl, and carbocyclyl, wherein:
the C₁₋₆alkyl, aryl, heteroaryl, heterocyclyl, or carbocyclyl is optionally substituted with one or more R⁷;
R⁶ is halogen;
R⁷ is selected from halogen, C₁₋₆alkyl, SO₂C₁₋₃alkyl, OC₁₋₃alkyl, OC₁₋₃haloalkyl, C₁₋₃alkylOH, C₁₋₃alkylNR⁸R⁹, OH, cyano, and C(O)OC₁₋₃alkyl, wherein:
the C₁₋₆alkyl, SO₂C₁₋₃alkyl, OC₁₋₃alkyl, OC₁₋₃haloalkyl, C₁₋₃alkylOH, C₁₋₃alkylNR⁸R⁹, or C(O)OC₁₋₃alkyl is optionally substituted with one or more R¹⁰;
as to R⁸ and R⁹:
R⁸ and R⁹ are independently selected from hydrogen, C₁₋₆alkyl, C₁₋₃alkylNR¹¹R¹², C₁₋₃alkylOaryl, heteroaryl, heterocyclyl, and carbocyclyl, wherein:
the C₁₋₆alkyl, C₁₋₃alkylNR¹¹R¹², C₁₋₃alkylOaryl, heteroaryl, heterocyclyl, or carbocyclyl is optionally substituted with one or more R¹⁰; or
R⁸ and R⁹, together with the atom to which they are attached, form a 4 to 6 membered ring; and
m is selected from 0 and 1.

4. A compound or pharmaceutically acceptable salt thereof according claim 1, wherein A is heteroaryl.

5. A compound or pharmaceutically acceptable salt thereof according to claim 4, wherein A is selected from pyridinyl and pyrimidinyl.

6. A compound or pharmaceutically acceptable salt thereof according claim 1, wherein A is aryl.

7. A compound or pharmaceutically acceptable salt thereof according to claim 6, wherein A is phenyl.

8. A compound or pharmaceutically acceptable salt thereof according claim 1, wherein A is selected from aryl and heteroaryl.

9. A compound or pharmaceutically acceptable salt thereof according claim 1, wherein A is selected from aryl and heteroaryl, wherein:
the aryl or heteroaryl is substituted with one or more R⁵.

10. A compound or pharmaceutically acceptable salt thereof according claim 1, wherein Z is selected from aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆haloalkyl, C₃₋₆cyclohaloalkyl, C₁₋₆alkylC₃₋₆cyclohaloalkyl, and C₁₋₆alkylheterocyclyl.

11. A compound or pharmaceutically acceptable salt thereof according to claim 1, wherein Z is selected from aryl, heteroaryl, heterocyclyl, C₃₋₆cycloalkyl, C₁₋₆alkyl, C₁₋₆haloalkyl, C₃₋₆cyclohaloalkyl, C₁₋₆alkylC₃₋₆cyclohaloalkyl, C₁₋₆alkylaryl, C₁₋₆alkylC₃₋₆cyclo alkyl, C₁₋₆alkylheteroaryl, and C₁₋₆alkylheterocyclyl.

12. A compound or pharmaceutically acceptable salt thereof according claims 1, wherein Z is selected from aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl, and C₂₋₆alkynylheterocyclyl.

13. A compound or pharmaceutically acceptable salt thereof according claim 1, wherein Z is selected from aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl, and C₂₋₆alkynylheterocyclyl, wherein:
the aryl, heteroaryl, heterocyclyl, C₃₋₆cycloalkyl, C₃₋₆cycloalkenyl, C₁₋₆alkyl, C₁₋₆alkylaryl, C₁₋₆alkylC₃₋₆cycloalkyl, C₁₋₆alkylheteroaryl, C₁₋₆alkylheterocyclyl, C₂₋₆alkenylaryl, C₂₋₆alkenyl, C₂₋₆alkenylC₃₋₆cycloalkyl, C₂₋₆alkenylheteroaryl, C₂₋₆alkenylheterocyclyl, C₂₋₆alkynylC₃₋₆cycloalkyl, C₂₋₆alkynyl, C₁₋₆haloalkyl, C₃₋₆cyclohaloalkyl, C₁₋₆alkylC₃₋₆cyclohaloalkyl, C₂₋₆alkynylaryl, C₂₋₆alkynylheteroaryl, or C₂₋₆alkynylheterocyclyl is substituted with one to three R⁷.

14. A compound or pharmaceutically acceptable salt thereof according to claim 13, wherein R⁷ is selected from halogen, C₁₋₆alkyl, SO₂C₁₋₃alkyl, OC₁₋₃alkyl, OC₁₋₃haloalkyl, and cyano, wherein:
the C₁₋₆alkyl, SO₂C₁₋₃alkyl, OC₁₋₃alkyl, or OC₁₋₃haloalkyl is optionally substituted with one or more R¹⁰.

15. A compound or pharmaceutically acceptable salt thereof according claim 1, wherein R⁶ is fluoro.

16. A compound or pharmaceutically acceptable salt thereof according claim 1, wherein m is 0.

17. A compound or pharmaceutically acceptable salt thereof according to claim 1, wherein:
R⁶ is halogen;
R⁷ is selected from halogen, C₁₋₆alkyl, OC₁₋₃alkyl, OC₁₋₃haloalkyl and cyano, wherein:
the C₁₋₆alkyl, OC₁₋₃alkyl, or OC₁₋₃haloalkyl is optionally substituted with one or more R¹⁰;
R¹⁰ is halo; and
m is selected from 0 and 1.

18. A compound or pharmaceutically acceptable salt thereof according to claim 1, wherein
A is heteroaryl optionally substituted with one or more R⁵;
B is aryl;
Z is selected from C₃₋₆cycloalkyl, C₁₋₆alkyl, and C₁₋₆alkylC₃₋₆cycloalkyl, wherein:
the C₃₋₆cycloalkyl, C₁₋₆alkyl, or C₁₋₆alkylC₃₋₆cycloalkyl is optionally substituted with one to three R⁷;
R⁵ is selected from C₁₋₆alkyl and OC₁₋₆alkyl, wherein:
the C₁₋₆alkyl or OC₁₋₆alkyl is optionally substituted with one to three R⁷;
R⁶ is halogen;
R⁷ is halogen; and
m is 0.

19. A compound or pharmaceutically acceptable salt thereof according to claim 17, wherein B is phenyl.

20. A compound or a pharmaceutically acceptable salt thereof, wherein the compound is selected from:
5-(3-Isobutoxyphenyl)-5-(pyridin-4-yl)-5H-pyrrolo[3,4-b]pyridin-7-amine;
5-(3-(Isopentyloxy)phenyl)-5-(pyridin-4-yl)-5H-pyrrolo[3,4-b]pyridin-7-amine;
5-(3-(Cyclopentylmethoxy)phenyl)-5-(pyridin-4-yl)-5H-pyrrolo[3,4-b]pyridin-7-amine;
5-(3-(Cyclobutylmethoxy)phenyl)-5-(pyridin-4-yl)-5H-pyrrolo[3,4-b]pyridin-7-amine;
5-(3-((2,2-Difluorocyclopropyl)methoxy)phenyl)-5-(pyridin-4-yl)-5H-pyrrolo[3,4-b]pyridin-7-amine;
5-(3-(3-Fluoropropoxy)phenyl)-5-(2-(trifluoromethyl)pyridin-4-yl)-5H-pyrrolo[3,4-b]pyridin-7-amine;
5-(3-(Cyclobutylmethoxy)phenyl)-5-(2-(trifluoromethyl)pyridin-4-yl)-5H-pyrrolo[3,4-b]pyridin-7-amine;
5-(3-(Cyclopentyloxy)phenyl)-5-(pyridin-4-yl)-5H-pyrrolo[3,4-b]pyridin-7-amine;
5-(3-Cyclobutoxyphenyl)-5-(pyridin-4-yl)-5H-pyrrolo[3,4-b]pyridin-7-amine;
5-(3-(3-Fluoropropoxy)phenyl)-5-(pyridin-4-yl)-5H-pyrrolo[3,4-b]pyridin-7-amine;
5-(3-(3-Fluoropropoxy)phenyl)-5-(4-methoxy-3,5-dimethylphenyl)-5H-pyrrolo[3,4-b]pyridin-7-amine;
5-(3-(3-Fluoropropoxy)phenyl)-5-(2-methoxypyridin-4-yl)-5H-pyrrolo[3,4-b]pyridin-7-amine;
5-(3-(3-Fluoropropoxy)phenyl)-5-(2-methylpyridin-4-yl)-5H-pyrrolo[3,4-b]pyridin-7-amine;
5-(3-(3-Fluoropropoxy)phenyl)-5-(4-methoxy-3-(trifluoromethyl)phenyl)-5H-pyrrolo[3,4-b]pyridin-7-amine;
5-(2,6-Dimethylpyridin-4-yl)-5-(3-isobutoxyphenyl)-5Hpyrrolo[3,4-b]pyridin-7-amine acetate;
5-(3-Isobutoxyphenyl)-5-(6-methoxy-5-methylpyridin-3-yl)-5H-pyrrolo[3,4-b]pyridin-7-amine acetate;
5-(3-Isobutoxyphenyl)-5-(6-methoxy-5-methylpyridin-3-yl)-5H-pyrrolo[3,4-b]pyridin-7-amine acetate;
5-(4-(Difluoromethoxy)phenyl)-5-(3-isobutoxyphenyl)-5H-pyrrolo[3,4-b]pyridin-7-amine;
5-(3-Methoxyphenyl)-5-(pyridin-4-yl)-5H-pyrrolo[3,4-b]pyridin-7-amine;
5-(2-(Difluoromethoxy)-6-methylpyridin-4-yl)-5-(3-(3-fluoropropoxy)phenyl)-5H-pyrrolo[3,4-b]pyridin-7-amine;
5-(3-Chloro-4-methoxyphenyl)-5-(3-(3-fluoropropoxy)phenyl)-5H-pyrrolo[3,4-b]pyridin-7-amine;
5-(3-(3,3-Difluoropropoxy)phenyl)-5-(2-(trifluoromethyl)pyridin-4-yl)-5H-pyrrolo[3,4-b]pyridin-7-amine;
5-(3-(3,3-Difluoropropoxy)phenyl)-5-(5-methoxy-4,6-dimethylpyridin-2-yl)-5H-pyrrolo[3,4-b]pyridin-7-amine; and
5-(2-(Difluoromethoxy)pyridin-4-yl)-5-(3-(3-fluoropropoxy)phenyl)-5H-pyrrolo[3,4-b]pyridin-7-amine.

21. A pharmaceutical composition, wherein the composition comprises:
a therapeutically effective amount of a compound or pharmaceutically acceptable salt thereof according claim 1; and
a pharmaceutically acceptable excipient, carrier, or diluent.

22-28. (canceled)

29. A method of inhibiting activity of BACE, wherein the method comprises contacting BACE with a compound or pharmaceutically acceptable salt thereof according to claim 1.

30. A method of treating or preventing an Aβ-related pathology in a mammal, wherein the method comprises administering to the mammal a therapeutically effective amount of a compound or pharmaceutically acceptable salt thereof according to claim 1.

31. The method of claim 30, wherein the Aβ-related pathology is selected from Downs syndrome, a β-amyloid angiopathy, cerebral amyloid angiopathy, hereditary cerebral hemorrhage, a disorder associated with cognitive impairment, mild cognitive impairment, Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy, and cortical basal degeneration.

32. A method of treating or preventing Alzheimer's Disease in a mammal, wherein the method comprises administering to the mammal a therapeutically effective amount of a compound or pharmaceutically acceptable salt thereof according to claim 1.

33. The method of claim 30, wherein the mammal is a human.

34. A method of treating or preventing an Aβ-related pathology in a mammal, wherein the method comprises administering to the mammal:
a therapeutically effective amount of a compound or pharmaceutically acceptable salt thereof according to claim 1; and
a one cognitive enhancing agent, memory enhancing agent, or choline esterase inhibitor.
